SEQUENCE LISTING (39 sequences)

SEQ ID NO: 1 - protein, 461 aa, Arabidopsis thaliana
MKVTQKPKIIFIPYPAQGHVTPMLHLASAFLSRGFSPVVMTPESIHRRISATNEDLGITFLALSDGQDRPDAPPSDFFSIENSMENIMPPQLERLLLEEDLDVACVVVDLLASWAIGVADRCGVPVAGFWPVMFAAYRLIQAIPELVRTGLVSQKGCPRQLEKTIVQPEQPLLSAEDLPWLIGTPKAQKKRFKFWQRTLERTKSLRWILTSSFKDEYEDVDNHKASYKKSNDLNKENNGQNPQILHLGPLHNQEATNNITITKTSFWEEDMSCLGWLQEQNPNSVIYISFGSWVSPIGESNIQTLALALEASGRPFLWALNRVWQEGLPPGFVHRVTITKNQGRIVSWAPQLEVLRNDSVGCYVTHCGWNSTMEAVASSRRLLCYPVAGDQFVNCKYIVDVWKIGVRLSGFGEKEVEDGLRKVMEDQDMGERLRKLRDRAMGNEARLSSEMNFTFLKNELN

SEQ ID NO: 2 - protein, 511 aa, Zea mays
MGAEAVAYPAVLLVPFPAQGHITPMLQLAGVLAAHGVAPTVALPDFIHRRIVAACGGGGVVGVTLASIPSGIDIVQQDAAAGDDDDTPGFRDIVHSMEHHMPLHLERMLTSPRRPPVACVVVDVLASWAVPVAARCGVPAAGFWPAMLACYRVVAAIPELLEKGLISESGTPISSSSTDSDEQDARTVRGLHILPAQVELRVEELPWLVGDSATRRSRFAFWLQTLHRARGLRWVLVNSFPAEAGCPAAAAAAAGDEDEDDGAHRQQGPRVIPVGAALLPAGGIGERTKQQQQCVNINKSPSMWRADSTCIGWLDAQRARSVVYVSFGSWVGSIGPGKVRELALGLEATGRPFLWALKRDPSWRAGLPDGFAGRVAGRGKLVDWAPQQDVLRHAAVGCYLTHCGWNSTLEAIQHGVRLLCYPVSGDQFINCAYITGLWKIGLRLGGMMRDDVRAGVERVMDDGGHLQEKVWALRERVVTPEARRGADRNVRSFVDEITRDHPLVVQLYSVL

SEQ ID NO: 3 - DNA, 24 bp, Artificial Sequence
  Primer
aaagtgtcctggcttgcagatacc

SEQ ID NO: 4 - DNA, 24 bp, artificial sequence
  primer
aagcattctagggcacacattgat

SEQ ID NO: 5 - DNA, 23 bp, artificial sequence
  primer
aaggctcgtggcatacctgtagt

SEQ ID NO: 6 - DNA, 21 bp, artificial sequence
  primer
gctgtacgtacgggtgcaatg

SEQ ID NO: 7 - DNA, 22 bp, artificial sequence
  primer
gtcatcactcatcaatcccagc

SEQ ID NO: 8 - DNA, 24 bp, artificial sequence
  primer
tcaacccccacctctctatttata

SEQ ID NO: 9 - DNA, 24 bp, artificial sequence
  primer
cctacccgctacaactggacataa

SEQ ID NO: 10 - DNA, 24 bp, artificial sequence
  primer
cagtactcgtttgtgcagtttgct

SEQ ID NO: 11 - DNA, 18 bp, artificial sequence
  primer
ctggtccgagatgatggc

SEQ ID NO: 12 - DNA, 20 bp, artificial sequence
  primer
tccatttctgcatctgcaac

SEQ ID NO: 13 - DNA, 22 bp, artificial sequence
  primer
ctctctttccttccgactttcc

SEQ ID NO: 14 - DNA, 19 bp, artificial sequence
  primer
gagcgagcgagagagatcg

SEQ ID NO: 15 - DNA, 24 bp, artificial sequence
  primer
ataaaacgaacgactctctcaccg

SEQ ID NO: 16 - DNA, 24 bp, artificial sequence
  primer
atatgtctgacgagcttcgacacc

SEQ ID NO: 17 - DNA, 22 bp, artificial sequence
  primer
gacgcgacttattcagcaccac

SEQ ID NO: 18 - DNA, 22 bp, artificial sequence
  primer
attgtttcagcgctgccggtta

SEQ ID NO: 19 - DNA, 23 bp, artificial sequence
  primer
caacttcgaggcagttcgtttat

SEQ ID NO: 20 - DNA, 24 bp, artificial sequence
  primer
agctcttgttgcaggaagtaggac

SEQ ID NO: 21 - DNA, 24 bp, artificial sequence
  primer
gcgttgtttggtagatcgttagcc

SEQ ID NO: 22 - DNA, 24 bp, artificial sequence
  primer
catatgcatcaggtcaagcaagga

SEQ ID NO: 23 - DNA, 24 bp, artificial sequence
  primer
actgcatctcacttgtcaccgtct

SEQ ID NO: 24 - DNA, 24 bp, artificial sequence
  primer
tgcagcttaaatttcatggacgtg

SEQ ID NO: 25 - DNA, 21 bp, artificial sequence
  primer
gccgaggatttcctgctgaag

SEQ ID NO: 26 - DNA, 24 bp, artificial sequence
  primer
gctcatgttgcttcacaacctctc

SEQ ID NO: 27 - DNA, 38 bp, artificial sequence
  primer
acactctttccctacacgacgctcttccgatctagctt

SEQ ID NO: 28 - DNA, 36 bp, artificial sequence
  primer
agctagatcggaagagcgtcgtgtagggaaagagtg

SEQ ID NO: 29 - DNA, 38 bp, artificial sequence
  primer
acactctttccctacacgacgctcttccgatctgctat

SEQ ID NO: 30 - DNA, 36 bp, artificial sequence
  primer
tagcagatcggaagagcgtcgtgtagggaaagagtg

SEQ ID NO: 31 - DNA, 38 bp, artificial sequence
  primer
acactctttccctacacgacgctcttccgatctctagt

SEQ ID NO: 32 - DNA, 36 bp, artificial sequence
  primer
ctagagatcggaagagcgtcgtgtagggaaagagtg

SEQ ID NO: 33 - DNA, 38 bp, artificial sequence
  primer
acactctttccctacacgacgctcttccgatctgatgt

SEQ ID NO: 34 - DNA, 36 bp, artificial sequence
  primer
catcagatcggaagagcgtcgtgtagggaaagagtg

SEQ ID NO: 35 - DNA, 33 bp, artificial sequence
  primer
ctcggcattcctgctgaaccgctcttccgatct

SEQ ID NO: 36 - DNA, 32 bp, artificial sequence
  primer
gatcggaagagcggttcagcaggaatgccgag

SEQ ID NO: 37 - DNA, 58 bp, artificial sequence
  primer
aatgatacggcgaccaccgagatctacactctttccctacacgacgctcttccgatct

SEQ ID NO: 38 - DNA, 61 bp, artificial sequence
  primer
caagcagaagacggcatacgagatcggtctcggcattcctgctgaaccgctcttccgatct

SEQ ID NO: 39 - protein, 44 aa, Arabidopsis thaliana
WAPQQDVLRHAAVGCYLTHCGWNSTLEAIQHGVRLLCYPVSGDQ

(19) United States
(12) Patent Application Publication
(10) Pub. No.: US 2021/0277410 A1
(43) Pub. Date: Sep. 9, 2021

(54) TRANSGENE AND MUTATIONAL CONTROL OF SEXUALITY IN MAIZE AND RELATED GRASSES

(71) Applicants: YALE UNIVERSITY, New Haven, CT (US); RHODE ISLAND COUNCIL ON POSTSECONDARY EDUCATION, Warwick, RI (US)

(72) Inventors: Stephen L. DELLAPORTA, Branford, CT (US); Andrew HAYWARD, Madison, CT (US); Maria MORENO, Branford, CT (US); Albert KAUSCH, Stonington, CT (US); John MOTTINGER, North Kingstown, RI (US)

(21) Appl. No.: 16/326,490

(22) PCT Filed: Aug. 11, 2017
(86) PCT No.: PCT/US17/46509

§ 371 Date: Feb. 19, 2019

Related U.S. Application Data
(60) Provisional application No. 62/377,088, filed on Aug. 19, 2016.

(51) Int. Cl. C12N 15/82

(52) U.S. Cl. CPC C12N 15/8216; C12N 15/8212; C12N 15/829; C12N 15/8289; C12N 15/8213

(57) ABSTRACT

The present invention pertains to genetically modified plants, particularly maize, sorghum and rice, with an all pistillate or all staminate phenotype and methods of the same. The survival of functional pistils in maize requires the action of the sk1 gene. SK1 encodes a glycosyltransferase (GT) that protects pistils from tasselseed-mediated cell death. sk1-dependent pistil protection at a developing floret gives rise to stamen arrest at the same floret, and so determines the pistillate floral fate. This is the first single gain-of-function gene known to control sexuality. The present invention further provides a direct strategy to extend hybrid technologies to related cereals such as sorghum and rice. Tasselseed and silkless genes represent major sex determination genes in maize, a pathway that permits the efficient production of hybrid seed and the associated benefits of heterosis-increased yield, resistance to pathogens, etc. Except for maize, current hybrid systems in cereals are fraught with genetic and environmental limitations. Genotype-independent hybrid cereal technology could potentially increase crop yields as much as 20-40% without placing additional land under agricultural production. This has profound implications for food security and the environmental impact of agriculture in some of the poorest regions of the world.

### CROSS REFERENCE TO RELATED APPLICATIONS

The present application claims priority under 35 U.S.C. § 119(e) to U.S. Provisional Patent Application No. 62/377,088, filed Aug. 19, 2016 which is incorporated herein by reference in its entirety.

### STATEMENT REGARDING FEDERALLY SPONSORED RESEARCH OR DEVELOPMENT

This invention was made with government support under grant numbers 0965420 and 1444478 awarded by the National Science Foundation and NIH/NCRR grant numbers RR019895 and RR029676. The government has certain rights in the invention.

### BACKGROUND OF THE INVENTION

The majority of flowering plants produce hermaphroditic flowers that contain both male (stamen) and female (pistil) reproductive organs. Certain flowering plants produce unisexual flowers, with staminate and pistillate flowers that arise on the same plant (monoecy) or on separate plants (dioecy). The staple crop Zea mays (maize) is monecious, producing a terminal staminate inflorescence called the tassel and axillary pistillate inflorescences called ears. Maize flowers (called florets in grasses) are characteristically arranged in paired spikelets each having two florets. The florets become staminate in the tassel through the selective elimination of a preformed pistil initial and sexual maturation of stamen. The ear spikelets become pistillate through the maturation of the pistil in the primary floret, and the arrest of all stamen initials in both florets. Unisexual flowers are highly advantageous for maize and other crop species, enabling hybrid production through outcrossing with progeny exhibiting heterosis while escaping inbreeding depression.

To generate staminate florets, the elimination of pistils requires a genetic pathway that include the tasselseed 1 and 2 (ts) genes. Mutant plants present a pistillate rather than staminate tassel and double pistils in the ear spikelets. The pistil elimination process involves the production of the phytohormone jasmonic acid (JA), which is dependent upon a TS1-encoded lipoxygenase localized to plant plastids and the activity of TS2, a short-chain alcohol dehydrogenase whose specific role in the signaling pathway remains elusive. The ts1 and ts2 genes are proposed to act in association with microRNAs miR156 and miR172 (ts4) to negatively regulate pistillate primary and secondary sex characteristics and promote staminate fate at the tassel inflorescence.

Unlike maize, other cereal crops including rice, wheat, sorghum, and millet, produce cosexual flowers that are both staminate and pistillate. These cosexual flowers are a strong impediment to the development and production of hybrid seed. A single gene system that produces unisexual flowers in these cereals is lacking. The application of such a system would permit a major improvement in the development of hybrid seed from these non-maize cereal crops. Accordingly, there is a long felt need for technology which facilitates the efficient production of hybrid seed from non-maize cereal crops. This need is partially satisfied by the following disclosure.

### SUMMARY OF THE INVENTION

In one aspect the invention provides an isolated polynucleotide encoding a polypeptide of SEQ ID NO: 2 or an amino acid sequence variant thereof operably linked to a heterologous promoter.

In various embodiments the heterologous promoter is a CaMV 35S promoter.

In various embodiments the isolated polynucleotide further comprises a marker gene. In various embodiments the marker gene is an herbicide resistance gene.

In various embodiments the herbicide resistance gene is bar.

In various embodiments the herbicide resistance gene encodes 5-enolpyruvyl-shikimate synthase (ESPS).

In various embodiments the marker gene affects the visual appearance of the seed or seedling.

In various embodiments the marker gene controls the appearance or distribution of anthrocyanin pigments in the seed or seedling.

In various embodiments, the invention provides a plant cell transformed with the isolated polynucleotide.

In another aspect, the invention provides a genetically modified plant comprising a transgene containing an sk1-encoded glycosyltransferase operably linked to a promoter for heterologous expression in the cells of the plant.

In various embodiments the plant is maize, sorghum or rice.

In various embodiments the genetically modified plant is a unisexual plant.

In another aspect, the invention provides a genetically modified plant comprising a transgene encoding a uridine diphosphate (UDP) glycosyltransferase.

In various embodiments the plant is maize, sorghum or rice.

In various embodiments the genetically modified plant comprises inflorescences of the pistillate phenotype associated with sk1.

In various embodiments the inflorescences are solely of the pistillate phenotype associated with sk1.

In another aspect, the invention provides a genetically modified plant comprising a mutation or transgene targeting an endogenous UDP glycosyltransferase and disrupting its activity.

In various embodiments the UDP glycosyltransferase is sk1.

In various embodiments the plant is maize, sorghum or rice.

In various embodiments the genetically modified plant comprises inflorescences of the staminate phenotype associated with the disruption of sk1.

In various embodiments the genetically modified plant is a unisexual plant.

In various embodiments the mutation is engineered using a CRISPR/Cas9 system.

In another aspect, the invention provides a method of generating a genetically modified plant comprising transforming a cell with a construct comprising a transgene encoding a UDP glycosyltransferase, thereby promoting the expression of the UDP glycosyltransferase in one or more cells of the plant.

In various embodiments the transgene is sk1.

In various embodiments the transgene comprises a polynucleotide encoding a polypeptide of SEQ ID NO: 2 or an amino acid sequence variant thereof.

In various embodiments the transgene is operably linked to a heterologous promoter.

In various embodiments the heterologous promoter is a CaMV 35S promoter.

In various embodiments the UDP glycosyltransferase localizes to a peroxisome.

In various embodiments the construct further comprises a marker gene.

In various embodiments the marker gene is an herbicide resistance gene.

In various embodiments the herbicide resistance gene is bar.

In various embodiments the herbicide resistance gene encodes 5-enolpyruvyl-shikimate synthase (ESPS).

In various embodiments the marker gene affects the visual appearance of a seed or seedling.

In various embodiments the marker gene controls the appearance or distribution of one or more anthrocyanin pigments in the seed or seedling.

In various embodiments the method further comprises using the marker gene to select at least one genetically modified plant.

In various embodiments the method further comprises using the genetically modified plant to generate a hybrid seed.

In various embodiments the plant is maize, rice or sorghum.

In another aspect, the invention provides a method of generating a transgenic plant comprising the step of engineering a mutation or transgene targeting an endogenous UDP glycosyltransferase and disrupting its activity.

In various embodiments the UDP glycosyltransferase is sk1.

In various embodiments the plant is maize, sorghum or rice.

In various embodiments the plant comprises at least one inflorescence of the staminate phenotype associated with the disruption of sk1.

In various embodiments the transgenic plant is a unisexual plant.

In various embodiments the mutation is engineered using a CRISPR/Cas9 system.

In another aspect, the invention provides a method of generating a transgenic plant comprising engineering a mutation in a 5′ or 3′ regulatory element of an endogenous UDP glycosyltransferase to alter an expression level of the UDP glycosyltransferase.

In various embodiments the transgenic plant is maize, rice or sorghum.

In various embodiments the transgenic plant is a unisexual plant.

In various embodiments the mutation is engineered using a crispr/Cas9 system, zinc-finger nucleases or transcription activator-like effects.

### BRIEF DESCRIPTION OF THE DRAWINGS

The following detailed description of preferred embodiments of the invention will be better understood when read in conjunction with the appended drawings. For the purpose of illustrating the invention, there are shown in the drawings embodiments which are presently preferred. It should be understood, however, that the invention is not limited to the precise arrangements and instrumentalities of the embodiments shown in the drawings.

FIG. 1A is a series of images illustrating a comparison of wild type ears (sk1/sk1-ref; left panels) and silkless 1 mutant ears (sk1-ref/sk1-ref; right panels). Left panels show respective ears at 2.5 cm in length, middle panels provide higher magnification to resolve individual spikelets, right panels show ears at 8 cm.

FIG. 1B is a Manhattan plot showing the depth of TaqαI read coverage (blue vertical lines) by chromosome position. Each x-axis pixel represents a bin of 1 MB and the logarithmic y-axis denotes the number of reads mapping to each bin. The sk1 genetic region (see FIG. 4) is shown enlarged with the TaqαI read coverage, Mu junction fragments (inverted triangles), and location of predicted and known genes.

FIG. 1C is an illustration of the structure of the sk1 gene, mutant alleles and protein motifs. Filled boxes at left and right indicate the 5′ and 3′ untranslated regions (UTRs), respectively. Open boxes indicate coding regions and angled lines indicate the single intron position. Insertions found in three sk1 mutant alleles are represented by inverted triangles positioned at the corresponding insertion site (see Table 1). The uridine diphosphate (UDP) glycosyltransferase signature/plant secondary product glycosyltransferase (PSPG) box is shown. The C-terminal 10 amino acids contain a PTS1-like domain is shown.

FIG. 1D is a WebLogo displaying the weighed alignment of the PSPG box of 107 identified Arabidopsis UGTs using ClustalW alignment. Conserved residues implicated in UDP-sugar binding are indicated with asterisks. The PSPG box of SK1 is shown below the WebLogo.

FIG. 1E is an illustration of the Maximum likelihood tree of SK1 homologs and the Arabidopsis UGT family. SK1 and its homologs cluster with the UGT Group N protein UGT82A1. Group N UGTs are indicated in red and bootstrapping confidence values shown at nodes.

FIG. 2A is a graph illustrating the mean sk1-B73 expression in maize tissues as determined by meta-analysis of RNA-seq datasets. Expression of sk1 in the shoot apical meristem (SAM), anthers, immature tassel, meiotic tassel, immature cob, pre-pollination cob, primary root, and eighth leaf. Error bars denote standard error. Normalized pseudo-read count determined as described in Methods.

FIG. 2B is an image demonstrating that Citrine:SVL colocalizes with a peroxisomal marker when transiently coexpressed in N. benthamiana. Scale bar is 20 μm. Insets show higher magnification.

FIG. 2C is an image demonstrating that SK1:Citrine localizes to the cytoplasm and does not colocalize with a peroxisomal marker when transiently coexpressed in N. benthamiana. Scale bar is 20 μm. Insets show higher magnification.

FIG. 2D is an image demonstrating that Citrine:SK1 colocalizes with a peroxisome marker when transiently expressed in N. benthamiana. Scale bar is 20 μm. Insets show higher magnification.

FIG. 2E is an image demonstrating that SK1ΔSVL:Citrine:SVL colocalizes with a transiently expressed peroxisomal marker in stable transgenic SK1ΔSVL:Citrine:SVL N. benthamiana. Scale bar is 20 μm. Insets show higher magnification.

FIG. 3A is an illustration of wild type maize terminal inflorescence (left) and 35S::SK1ΔSVL:Citrine:SVL maize terminal inflorescence (right). The 35S::SK1ΔSVL:Citrine:SVL transgenic maize T0 plants display a pistillate phenotype where the tassel inflorescence is completely feminized.

FIG. 3B is an illustration of representative T1 plants segregating for the presence and absence of the 35S::SK1ΔSVL:Citrine:SVL transgene. All plants displaying a pistillate phenotype tested positive for the presence of the transgene (see FIG. 7).

FIG. 3C is a box plot summarizing the distribution of OPDA and JA in T1 plants segregating for the 35S::SK1ΔSVL:Citrine:SVL transgene. Jasmonates were measured in the staminate terminal inflorescence of plants without the transgene (+/+) and in the pistillate terminal inflorescence of plants containing the 35S::SK1ΔSVL:Citrine:SVL transgene (SK1-CIT/+). Open circles represent individual measurements. Whiskers extend to minimum and maximum values.

FIG. 4A is a diagram depicting a genetic map interval of sk1. The number of recombination breakpoints is shown below each marker.

FIG. 4B is a diagram showing a refined map interval of sk1.

FIG. 4C is a map of the genomic region of sk1 on Chromosome 2 showing positions of flanking markers used to define the sk1 genetic interval. The approximate location of GRMZM2G021768 at Chr2:27,602,064 . . . 27,606,189 is also shown. All positions are based on B73 RefGen_v3 available at maizegdb dot org).

FIG. 5A is a Bayesian unrooted tree of the five most highly related SK1 proteins from B. dystachion, O. sativa, S. italica, S. bicolor, and Z. mays. Genes clustering with SK1 (GRMZM2G021786), were retained for further analysis.

FIG. 5B is a Bayesian rooted tree containing putative SK1 homologs from B. dystachion, O. sativa, S. italica, S. bicolor, and Z. mays. The Arabidopsis nearest hit to SK1, AT3G22250.1 (UGT82A1), was used as the outgroup. Bayesian posterior probabilities are indicated at each node.

FIG. 5C is a Clustal Omega amino acid alignment of Arabidopsis AT3G22250.1 (UGT82A1) and maize SK1 (GRMZM2G021786). Position of conserved amino acids indicated as fully conserved (*), strongly similar amino acids (:) with Gonnet PM250 matrix score>0.5, and weakly similar (.) with score=<0.5. Clustal Omega v1.2.1 may be found at ebi dot ac dot uk/Tools/msa/clustalo/.

FIG. 6A is an image generated by fluorescence microscopy depicting Citrine:SVL localizing to punctate bodies when transiently expressed in N. benthamiana. Scale bar is 20 μm.

FIG. 6B is an image generated by fluorescence microscopy depicting SK1:Citrine showing diffuse cytoplasmic localization and not punctate localization when transiently expressed in N. benthamiana. Scale bar is 20 μm.

FIG. 6C is an image generated by fluorescence microscopy depicting SK1ΔSVL:Citrine:SVL localizing to punctate bodies in stable transgenic Arabidopsis leaf tissue. Scale bar is 20 μm.

FIG. 6D is an image generated by fluorescence microscopy depicting SK1ΔSVL:Citrine:SVL localizing to punctate bodies in stable transgenic N. benthamiana leaf tissue. Scale bar is 20 μm.

FIG. 6E is an image generated by fluorescence microscopy depicting SK1ΔSVL:Citrine:SVL localizes to punctate bodies in stable transgenic maize leaf tissue. Scale bar is 20 μm.

FIG. 6F is an image of Western blots that confirm expression of the fluorescent proteins described here and in FIG. 2. Asterisk indicates position of Citrine cleavage product.

FIG. 7A is a series of images of representative examples of the SK1ΔSVL:Citrine:SVL T0 plant screening process. Leaf tissue was screened for Citrine fluorescence using a Typhoon imager. A single plant from event E02 that was negative for Citrine fluorescence, shown here, was maintained and displayed a wild type staminate tassel phenotype. Plants positive for Citrine fluorescence (n=72), such as those from event E17 and event E42, were scored at flowering and all Citrine-positive plants developed a complete pistillate phenotype.

FIG. 7B is a series of images showing plants of the pistillate phenotype of SK1ΔSVL:Citrine:SVL T0 maize representing five independent transformation events.

FIG. 7C is a composite image of a gel and a diagram showing that the pistillate terminal inflorescence phenotype cosegregated perfectly with SK1ΔSVL:Citrine:SVL transgene. T0 SK1ΔSVL:Citrine:SVL plants were crossed to A188. Individual T1 progeny were scored for phosphinothricin herbicide resistance encoded by the physically linked selectable marker bar used in the transformation vector to determine the presence or absence of the SK1ΔSVL:Citrine:SVL transgene cassette. Plants were also scored for the presence of the SK1ΔSVL:Citrine:SVL transgene by PCR. The pistillate (pi) phenotype cosegregated perfectly with the presence of the SK1ΔSVL:Citrine:SVL transgene and the bar selectable marker.

FIG. 7D is a composite image of a gel and a diagram showing that the pistillate terminal inflorescence phenotype cosegregated perfectly with SK1ΔSVL:Citrine:SVL transgene. T0 SK1ΔSVL:Citrine:SVL plants were crossed to sk1-ref. Individual T1 progeny were scored for phosphinothricin herbicide resistance encoded by the physically linked selectable marker bar used in the transformation vector to determine the presence or absence of the SK1ΔSVL:Citrine:SVL transgene cassette. Plants were also scored for the presence of the SK1ΔSVL:Citrine:SVL transgene by PCR. The pistillate (pi) phenotype cosegregated perfectly with the presence of the SK1ΔSVL:Citrine:SVL transgene and the bar selectable marker.

FIG. 8A is an image depicting the staminate wild type tassel at 8 cm.

FIG. 8B is an image depicting the pistillate SK1ΔSVL:Citrine:SVL tassel at 8 cm.

FIG. 8C is an image of a spikelet from an SK1ΔSVL:Citrine:SVL tassel showing both upper and lower pistillate florets.

FIG. 8D is an image depicting a branch of an SK1ΔSVL:Citrine:SVL tassel displaying nearly complete penetrance of the pistillate phenotype. Only the most terminal florets display a cosexual phenotype.

FIG. 8E is an image depicting an example of a rare cosexual terminal spikelet from an SK1ΔSVL:Citrine:SVL tassel.

FIG. 8F is an image depicting a spikelet from an SK1ΔSVL:Citrine:SVL ear showing that both the upper and lower floret are pistillate.

### DETAILED DESCRIPTION

### The sk1 Gene

In one embodiment, the sk1 gene or coding sequence (CDS) thereof is synthetically engineered to be expressed from a heterologous promoter and transformed into a plant cell. Such heterologous promoters facilitate expression of sk1 constitutively throughout the plant or in a tissue-specific manner to block pistil death. One example of a suitable heterologous promoter is the CaMV 35S promoter. The invention should not be construed to be limited to this promoter in that any heterologous promoter that facilitates expression of sk1 to prevent pistil destruction should be considered to be included in the invention.

In another embodiment, there is provided an isolated DNA fragment comprising the coding region of the sk1-encoded glycosyltransferase from maize or closely related species, where the DNA is adapted for expression in plants, and therefore includes a suitable promoter for constitutive, tissue- or cell-specific expression. In one embodiment a transgene containing sk1-encoded glycosyltransferase is operably linked to a suitable promoter for heterologous expression in plants.

In yet another embodiment, the sk1 transgene is co-expressed with a marker gene to permit the identification of sk1 transgenic plants in the lab or the field. Non-limiting examples of marker genes are herbicide-resistance genes such as the bar or 5-enolpyruvyl-shikimate synthase (ESPS) genes that can be used as selectable markers in both the lab and the field. These stacked herbicide resistance and sk1 transgenes can be used as selectable markers in plant transformation experiments and because they co-segregate in progeny, would allow for the identification of sk1 transgenics in the field. This is useful for several purposes including, but not limited to: 1) cells transformed with sk1 transgenes can be identified using herbicides as selectable markers in tissue culture, in whole plants, or in field applications; 2) the herbicide can be used as a selection for plants containing the sk1 transgene in breeding new lines; and 3) herbicide application in the field can be used to select for sk1 transgenics in a population segregating for the transgene. The latter usage is especially important for the ability to create hybrid seed. Another example of a marker gene is one that can be visualized in the seed or seedling. In certain embodiments such marker genes can control the deposition of anthocyanin pigments in the seed or seedling.

### Expression of the sk1 Gene

In one embodiment, a mutation in an endogenous sk1 gene is generated so as to facilitate expression of this gene in a heterologous manner in plants. For example, a dominant gain of function allele of sk1 can be engineered by modifying the 5′ or 3′ regulatory sequences of endogenous sk1 in order to block pistil death in a floret that would not normally express sk1 without these modifications. This targeted modification of endogenous sk1 to generate a pistillate flower can be achieved using the CRISPR/Cas9 system, zinc-finger nucleases, transcription activator-like effector nucleases, or other technologies of this type well known to the skilled artisan.

In another embodiment a transgene targeting endogenous sk1 or a closely related glycosyltransferase in plants is used to disrupt its activity. In another embodiment, a mutation in endogenous sk1 is generated in order to knock down the expression of sk1 in its natural environment. For example, pistil destruction in a floret can be promoted by the targeted disruption of sk1 using the CRISPR/Cas9 system or other similar methods. The disruption of sk1 should result in an effective recessive mutation manifesting as staminate flowers in a homozygous plant.

### Methods of Using the Sk1 Gene

The present invention provides a novel and innovative approach to use heterologous expression of a maize sex determination gene, silkless1 (sk1), to achieve the production of unisexual flowers (staminate or pistillate) in maize and related cereals. The tasselseed genes, specifically ts1 and ts2 are required to eliminate pistils while permitting stamens to mature. Pistil elimination by tasselseed action results in completely staminate flowers (called florets in grasses such as maize and related cereals). The ts1 and ts2 gene products have been shown to cause pistil cell death through a jasmonic acid (JA) signaling pathway. In another embodiment a synthetic mutation in an endogenous or orthologous sk1 gene is engineered for the purposes of manipulating floral sexuality or endogenous jasmonate levels.

The invention further pertains to the application of the sk1-encoded glycosyltransferase as a method of manipulating the sexual fate of flowers. It has been discovered in the present invention that abolishing sk1 protection eliminates pistil formation in the florets. Similarly, it has been discovered in the present invention that expression of sk1 protects pistils from tasselseed-mediated elimination. The invention therefore includes the use of sk1 alone or in combination with tasselseed genes in a method of manipulating the sexual fate of florets.

Maize plants produce both staminate (“male”) and pistillate (“female”) unisexual flowers on a single plant. Specifically, a maize plant produces a primary apical staminate flower (the “tassel”) and one or more axillary pistillate flowers (the “ears”). Hybrid seed is produced by crossing staminate flowers from a selected genetic background to pistillate flowers from a second selected genetic background. There is currently no system that produces unisexual maize plants that are either completely staminate or completely pistillate in order to expedite the production of hybrid seed. Such a system would enable the rapid development of hybrid maize seed from novel genetic backgrounds. Such a system would also enable the expedited production of hybrid seed from previously established genetic backgrounds.

Accordingly, in another embodiment of the invention there is provided a genetically modified unisexual plant comprising a transgene encoding sk1 or a closely related UDP glycosyltransferase. In various embodiments, a method of producing hybrid seeds are provided where the method comprises the step of crossing unisexual plants generated by either the inclusion of an sk1 transgene or a closely related UDP glycosyltransferase or the disruption of endogenous sk1. In certain non-limiting aspects, the unisexual plants are cereal grains, for example maize, sorghum or rice.

In various embodiments, additional sk1-related glycosyltransferases with the same biological activity and synthetically engineered for peroxisome localization as sk1 are used to achieve the objectives described herein.

### Definitions

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

The term “abnormal” when used in the context of organisms, tissues, cells or components thereof, refers to those organisms, tissues, cells or components thereof that differ in at least one observable or detectable characteristic (e.g., age, treatment, time of day, etc.) from those organisms, tissues, cells or components thereof that display the “normal” (expected) respective characteristic. Characteristics which are normal or expected for one cell or tissue type, might be abnormal for a different cell or tissue type.

As used herein the terms “alteration,” “defect,” “variation” or “mutation” refer to a mutation in a gene in a cell that affects the function, activity, expression (transcription or translation) or conformation of the polypeptide it encodes. Mutations encompassed by the present invention can be any mutation of a gene in a cell that results in the enhancement or disruption of the function, activity, expression or conformation of the encoded polypeptide, including the complete absence of expression of the encoded protein and can include, for example, missense and nonsense mutations, insertions, deletions, frameshifts and premature terminations. Without being so limited, mutations encompassed by the present invention may alter splicing the mRNA (splice site mutation) or cause a shift in the reading frame (frameshift).

The term “amino acid sequence variant” refers to polypeptides having amino acid sequences that differ to some extent from a native sequence polypeptide. Ordinarily, amino acid sequence variants will possess at least about 70% homology, or at least about 80%, or at least about 90% homology to the native polypeptide. The amino acid sequence variants possess substitutions, deletions, and/or insertions at certain positions within the amino acid sequence of the native amino acid sequence.

As used herein, the term “binding” refers to the adherence of molecules to one another, such as, but not limited to, enzymes to substrates, antibodies to antigens, DNA strands to their complementary strands. Binding occurs because the shape and chemical nature of parts of the molecule surfaces are complementary. A common metaphor is the “lock-and-key” used to describe how enzymes fit around their substrate.

The term “coding sequence,” as used herein, means a sequence of a nucleic acid or its complement, or a part thereof, that can be transcribed and/or translated to produce the mRNA and/or the polypeptide or a fragment thereof. Coding sequences include exons in a genomic DNA or immature primary RNA transcripts, which are joined together by the cell's biochemical machinery to provide a mature mRNA. The anti-sense strand is the complement of such a nucleic acid, and the coding sequence can be deduced therefrom. In contrast, the term “non-coding sequence,” as used herein, means a sequence of a nucleic acid or its complement, or a part thereof, that is not translated into amino acid in vivo, or where tRNA does not interact to place or attempt to place an amino acid. Non-coding sequences include both intron sequences in genomic DNA or immature primary RNA transcripts, and gene-associated sequences such as promoters, enhancers, silencers, and the like.

As used herein, the terms “complementary” or “complementarity” are used in reference to polynucleotides (i.e., a sequence of nucleotides) related by the base-pairing rules. For example, the sequence “A-G-T,” is complementary to the sequence “T-C-A.” Complementarity may be “partial,” in which only some of the nucleic acids' bases are matched according to the base pairing rules. Or, there may be “complete” or “total” complementarity between the nucleic acids. The degree of complementarity between nucleic acid strands has significant effects on the efficiency and strength of hybridization between nucleic acid strands. This is of particular importance in amplification reactions, as well as detection methods that depend upon binding between nucleic acids.

As used herein, the terms “conservative variation” or “conservative substitution” as used herein refers to the replacement of an amino acid residue by another, biologically similar residue. Conservative variations or substitutions are not likely to change the shape of the peptide chain. Examples of conservative variations, or substitutions, include the replacement of one hydrophobic residue such as isoleucine, valine, leucine or methionine for another, or the substitution of one polar residue for another, such as the substitution of arginine for lysine, glutamic for aspartic acid, or glutamine for asparagine, and the like.

As used herein, the term “domain” refers to a part of a molecule or structure that shares common physicochemical features, such as, but not limited to, hydrophobic, polar, globular and helical domains or properties. Specific examples of binding domains include, but are not limited to, DNA binding domains and ATP binding domains.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

The term “expression” as used herein is defined as the transcription and/or translation of a particular nucleotide sequence driven by its promoter.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art that pertain to expression of genes in plant cells.

As used herein, the term “fusion peptide” or “fusion polypeptide” or “fusion protein” or “fusion peptidomimetic” or “fusion non-peptide-analog” refers to a heterologous peptide, heterologous polypeptide, heterologous protein, peptidomimetic, or non-peptide analog linked to a membrane translocation domain.

As used herein, the term “fragment,” as applied to a nucleic acid, refers to a subsequence of a larger nucleic acid. A “fragment” of a nucleic acid can be at least about 15 nucleotides in length; for example, at least about 50 nucleotides to about 100 nucleotides; at least about 100 to about 500 nucleotides, at least about 500 to about 1000 nucleotides; at least about 1000 nucleotides to about 1500 nucleotides; about 1500 nucleotides to about 2500 nucleotides; or about 2500 nucleotides (and any integer value in between). As used herein, the term “fragment,” as applied to a protein or peptide, refers to a subsequence of a larger protein or peptide. A “fragment” of a protein or peptide can be at least about 20 amino acids in length; for example, at least about 50 amino acids in length; at least about 100 amino acids in length; at least about 200 amino acids in length; at least about 300 amino acids in length; or at least about 400 amino acids in length (and any integer value in between).

“Homologous” refers to the sequence similarity or sequence identity between two polypeptides or between two nucleic acid molecules. When a position in both of the two compared sequences is occupied by the same base or amino acid monomer subunit, e.g., if a position in each of two DNA molecules is occupied by adenine, then the molecules are homologous at that position. The percent of homology between two sequences is a function of the number of matching or homologous positions shared by the two sequences divided by the number of positions compared ×100. For example, if 6 of 10 of the positions in two sequences are matched or homologous then the two sequences are 60% homologous. By way of example, the DNA sequences ATTGCC and TATGGC share 50% homology. Generally, a comparison is made when two sequences are aligned to give maximum homology.

A “nucleic acid” refers to a polynucleotide and includes poly-ribonucleotides and poly-deoxyribonucleotides. Nucleic acids according to the present invention may include any polymer or oligomer of pyrimidine and purine bases, preferably cytosine, thymine, and uracil, and adenine and guanine, respectively. See Albert L. Lehninger, Principles of Biochemistry, at 793-800 (Worth Pub. 1982) which is herein incorporated in its entirety for all purposes. Indeed, the present invention contemplates any deoxyribonucleotide, ribonucleotide or peptide nucleic acid component, and any chemical variants thereof, such as methylated, hydroxymethylated or glucosylated forms of these bases, and the like. The polymers or oligomers may be heterogeneous or homogeneous in composition, and may be isolated from naturally occurring sources or may be artificially or synthetically produced. In addition, the nucleic acids may be DNA or RNA, or a mixture thereof, and may exist permanently or transitionally in single-stranded or double-stranded form, including homoduplex, heteroduplex, and hybrid states.

An “oligonucleotide” or “polynucleotide” is a nucleic acid ranging from at least 2, in certain embodiments at least 8, 15 or 25 nucleotides in length, but may be up to 50, 100, 1000, or 5000 nucleotides long or a compound that specifically hybridizes to a polynucleotide. Polynucleotides include sequences of deoxyribonucleic acid (DNA) or ribonucleic acid (RNA) or mimetics thereof which may be isolated from natural sources, recombinantly produced or artificially synthesized. A further example of a polynucleotide of the present invention may be a peptide nucleic acid (PNA). (See U.S. Pat. No. 6,156,501 which is hereby incorporated by reference in its entirety) The invention also encompasses situations in which there is a nontraditional base pairing such as Hoogsteen base pairing which has been identified in certain tRNA molecules and postulated to exist in a triple helix. “Polynucleotide” and “oligonucleotide” are used interchangeably herein. It is understood that when a nucleotide sequence is represented herein by a DNA sequence (e.g., A, T, G, and C), this also includes the corresponding RNA sequence (e.g., A, U, G, C) in which “U” replaces T.

The term “promoter” as used herein is defined as a DNA sequence recognized by the synthetic machinery of the cell, or introduced synthetic machinery, required to initiate the specific transcription of a polynucleotide sequence.

As used herein, the term “promoter/regulatory sequence” means a nucleic acid sequence which is required for expression of a gene product operably linked to the promoter/regulatory sequence. In some instances, this sequence may be the core promoter sequence and in other instances, this sequence may also include an enhancer sequence and other regulatory elements which are required for expression of the gene product. The promoter/regulatory sequence may, for example, be one which expresses the gene product in a tissue specific manner.

A “constitutive” promoter is a nucleotide sequence which, when operably linked with a polynucleotide which encodes or specifies a gene product, causes the gene product to be produced in a cell under most or all physiological conditions of the cell.

An “inducible” promoter is a nucleotide sequence which, when operably linked with a polynucleotide which encodes or specifies a gene product, causes the gene product to be produced in a cell substantially only when an inducer which corresponds to the promoter is present in the cell.

A “tissue-specific” promoter is a nucleotide sequence which, when operably linked with a polynucleotide encodes or specified by a gene, causes the gene product to be produced in a cell substantially only if the cell is a cell of the tissue type corresponding to the promoter.

The phrase “under transcriptional control” or “operatively linked” as used herein means that the promoter is in the correct location and orientation in relation to a polynucleotide to control the initiation of transcription by RNA polymerase and expression of the polynucleotide.

As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation. Suitable methods for plants include the use of gold nanoparticles and the use of a viral vector such as Agrobacterium tumefaciens. Suitable methods for transforming or transfecting host cells can be found in Sambrook, et al. (Molecular Cloning: A Laboratory Manual. 2nd, ed., Cold Spring Harbor Laboratory, Cold Spring Harbor Laboratory Press, Cold Spring Harbor, N.Y., 1989), and other laboratory manuals.

As used herein, the term “wild-type” refers to the genotype and phenotype that is characteristic of most of the members of a species occurring naturally and contrasting with the genotype and phenotype of a mutant.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the compounds of the present invention and practice the claimed methods. The following working examples therefore, specifically point out the preferred embodiments of the present invention, and are not to be construed as limiting in any way the remainder of the disclosure.

The materials and methods employed in these experiments are now described.

### Materials and Methods

### Genetic Stocks

TABLE 1
sk1 alleles used in this study

| | | Target site | | Reference |
|---|---|---|---|---|
| Allele | Mutation | duplication | Position* | (source) |
| sk1-ref | >4 kb Helitron insertion | none | 1562 (intron | Jones et al. |
| | | | 1) | 1925 (Maize |
| | | | | Coop) |
| sk1- | 1379 bp Mu1 insertion | GCTGGCGCT | 2537 (exon 2) | Rescue Mu |
| rMu | | | | lines (Maize |
| | | | | Coop) |
| sk1- | 3549 bp uncharacterized | GTACA | 2544 (intron | This |
| Allie1 | insertion | | 1) | study |

*Based on B73 RefGen_v3 genomic DNA sequence, maizegdb dot org/gene_center/gene?id=GRMZM2G021786

The sk1-ref allele and the sk1-mu1 allele were obtained from the Maize Genetics Cooperation Stock Center (maizecoop dot cropsci dot uiuc dot edu). Several sk1 alleles were originally found as segregating silkless plants arising from the active Mutator lines from the RescueMu project. See J. Fernandes et al., Genome Biol. 5, R82 (2004). Because these plants were derived from a population of Mutator plants with common parents, it was likely that they represented a single recessive mutation herein referred to as the sk1-mu1 allele. To determine allelism with the sk1 reference allele, sk1-mu1/sk1-mu1 pollen was crossed to female Sk1-W22/sk1-ref plants. The progeny of this cross segregated 1:1 for silkless confirming that sk1-mu1 is allelic to sk1-ref. The sk1-Allie1 allele was recovered by test crossing sk1-ref/sk1-ref plants to females homozygous for b-Peru:dSpm with an active Spms. Kernels showing a high degree of instability were selected and planted. In a population of approximately 6000, three silkless mutant plants were found that failed to complement the sk1-ref mutation, including the sk1-Allie1 mutant.

### Selection and Design of Molecular Markers for Sk1-Ref Mapping

Molecular markers were initially selected from the IBM2 2004 neighbors genetic map of maize chromosome 2 available at www.maizegdb.org. This tool was developed by the Maize Mapping Project and at the time that this study began in 2009, it was the best resolved genetic map of maize with ˜2,000 loci. Table 2 presents a list of the markers used. In most cases, these were Simple Sequence Repeats (SSRs) that had previously been developed into PCR-based assays. In other cases, as noted in Table 2, the genomic sequence of the markers was obtained from W22 and sk1-ref lines and used to design CAPS (Cleaved Amplified Polymorphic Sequences) assays according to A. Konieczny, F. M. Ausubel, A procedure for mapping Arabidopsis mutations using co-dominant ecotype-specific PCR-based markers. Plant J. 4, 403-410 (1993). Additional CAPS markers were designed from predicted gene sequences previously filtered for repetitive DNA in the TIGR Maize Database.

### Physical Mapping of Sk1-Ref Genetic Interval

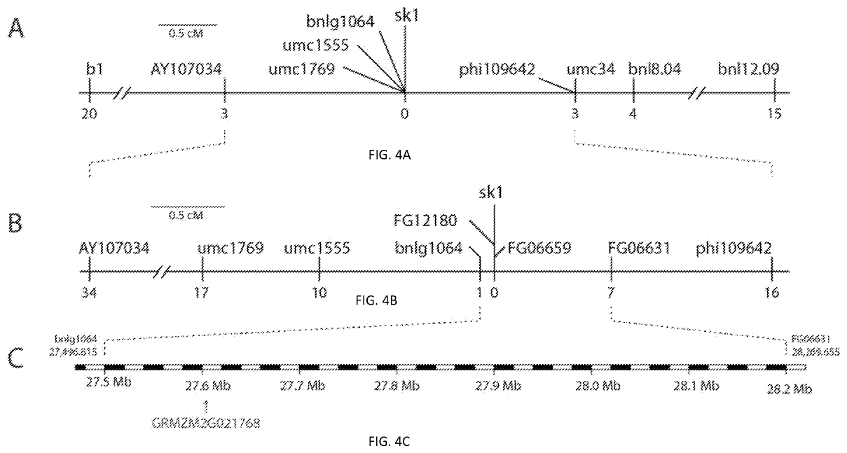

A physical map position of sk1 was initially defined utilizing a population of 198 testcross individuals segregating 1:1 for wild-type (sk1-ref/Sk1-W22) and mutant (sk1-ref/sk1-ref) plants. Molecular marker umc34 was identified as located proximal and the closest to sk1, at ˜1.5 cM (FIG. 4A). The search for distal markers to umc34 led to the creation of a CAPS marker from AY107034, an EST anchored in the maize physical map. This CAPS marker was tested in the initial recombinant population and three out of 20 distal recombination breakpoints were shown to map proximal to AY107034, which indicated that this was the closest distal marker to sk1, at ˜1.5 cM, identified so far (FIG. 4A). The flanking markers AY107034 and phi109642 (a SSR marker used as an alternative to umc34 because it showed complete linkage to umc34 in the mapping population and was simpler to score in genotyping experiments) were used in high-throughput screenings in a mapping population expanded to 634 individuals. This analysis resulted in the identification of 44 individuals showing a recombination breakpoint between these flanking markers. These recombinant individuals were later scored for the silkless phenotype at maturity. Markers umc1769, umc1555 and bn1g1064 were subsequently evaluated in all 44 recombinant individuals. The refined genetic map (FIG. 4B) was derived based on data from all mapping populations analyzed up to this point (n=832). The distal end of the sk1 genetic interval was delimited by bn1g1064 at 0.1 cM (1 crossover), while the proximal end marked by phi109642 remained at ˜1.9 cM from sk1 (FIG. 4A), refining the sk1 physical interval to ˜1 Mb. A closer sk1 proximal marker was sought among the predicted genes of the BAC clone Z377J20 available at that time. A CAPS marker from the predicted gene FG06631 was designed and tested in the 16 proximal sk1 recombinants. Seven of these recombinants mapped distal to FG06631, establishing this marker as the new proximal boundary in the sk1 genetic interval at 0.8 cM (FIG. 4B). This interval was found to contain 13 putative genes based upon the 2008 maize filtered gene set, a number that expanded to ˜30 upon the release of the B73 reference genome.

### Mu-Taq Library Construction and Identification of Sk1-Mu1

Genomic sk1-rMu1 DNA was digested with TaqαI, end-repaired, adenylated and ligated to custom Illumina paired-end adapters as described in T. P. Howard, 3rd et al., Identification of the maize gravitropism gene lazy plant1 by a transposon-tagging genome resequencing strategy. PloS one 9, e87053 (2014) with the modifications described below. TaqαI libraries were created with genomic DNA extracted from four independent plants homozygous for the sk1-rMu1 allele. The custom adaptors used for sk1-rMu1 cloning were of an earlier iteration than those described in Howard et al. One adapter incorporated a 4 bp barcode index while the other was a common adapter (Table 2). These adaptors were essentially identical to those described previously in Elshire et al., PloS one 6, e19379 (2011). Each genomic sample was associated with a unique barcoded adapter. 18 μl of end-repaired, adenylated TaqαI fragments were ligated to adaptors by 5 μl Quick T4 DNA Ligase (NEB) in 50 μl reactions containing 28 nM each of adapter in 1× Quick Ligase Buffer (NEB). Reactions were incubated at 20° C. for 20 minutes. Excess adaptors were removed using Microcon YM-50 columns (Millipore) as described in Howard et al. Duplicate 50 μl PCR reactions were performed to enrich each sample for sequencing. Reactions contained 1× Phusion High-Fidelity PCR Master Mix with HF Buffer (NEB), 500 nM of each primer (see Table 2), and ˜100 ng adapted DNA. Cycling instructions were as follows: 98° C. (2 minutes); 15 cycles of 98° C. (10 seconds), 65° C. (30 seconds), 72° C. (30 seconds); 72° C. (5 minutes). All barcoded, amplified samples were multiplexed (pooled) and the buffer exchanged to 1×TE using Microcon YM-30 as described in Howard et al. No gel extraction step or qPCR step was performed to normalize the concentrations of each sample before pooling. Sequencing was performed using an Illumina Genome Analyzer IIx at the Yale Center for Genome Analysis.

### Genome Walking and PCR-Based Fine Mapping of Sk1 Alleles

Identification and fine mapping of the sk1-mu1, sk1-ref, and sk1-Allie1 alleles was performed by PCR reaction using insertion-specific primer pairs with Phusion DNA polymerase. Identification of the Helitron-like insertion in sk1-ref plants was mediated by NaeI, SfoI and Stul Genomewalker (CLONTECH®) libraries using nested PCR reactions. PCR primers were designed based upon the B73-reference genome. Primers and PCR conditions used for the mapping of individual sk1 alleles are available upon request.

### Phylogenetic Analysis of Sk1

Phylogeny was determined using a two-step analysis. First, the top five most related proteins to SK1 (GRMZM2G021768) were determined by Blastp score under default Gramene settings (allowing some local misalignments) for B. dystachion, O. sativa, S. italica, S. bicolor, Z. mays. Analysis of two top hits from rice, Os04T0525100 and Os04T0525200, suggested that they were two exons of the same gene, and so these two sequences were combined into one for the final phylogeny (Os04T0525100-200). Amino acid sequences were aligned using the ClustalW module (BLOSUM Matrix, Gap open penality=3.0, Gap extension penalty=1.8) in MEGA6. See K. Tamura et al., Mol Biol Evol 30, 2725-2729 (2013). Regions with missing sequence were trimmed visually. A maximum likelihood method in MEGA was used to determine the optimal amino acid substitution model. An initial tree was built using MrBayes, as described in F. Ronquist, J. P. Huelsenbeck, MrBayes 3: Bayesian phylogenetic inference under mixed models, Bioinformatics 19, 1572-1574 (2003), using a Wheland and Goldman substitution model, four chains, heat 0.5, and 1,000,000 iterations. Final standard deviation of split frequencies was 0.002696. Genes separated into two general clusters in this tree (FIG. 5A). All genes in the SK1 cluster were retained for further analysis.

A second phylogenetic tree was generated to better quantify the relationships between sk1 and its homologs (FIG. 5B). This tree was generated with the coding sequences of selected monocot genes plus Arabidopsis gene AT3G22250, which was identified as the closest homolog to maize SK1 via Blastp and was set as an outgroup. Alignment was performed in MEGA6 via ClustalW (Codon alignment, gap open penalty 3.0, gap extension penalty 1.8). Aligned sequence was then trimmed visually to remove regions with excessive missing sequence. A nucleotide substitution model was selected using a maximum likelihood method in MEGA6. The final tree was built in MrBayes using a General Time Reversible Model with Gamma Distribution (1,000,000 iterations, 4 chains, temp 0.1, sumt burnin 1000). Final standard deviation of split frequencies was 0.003863.

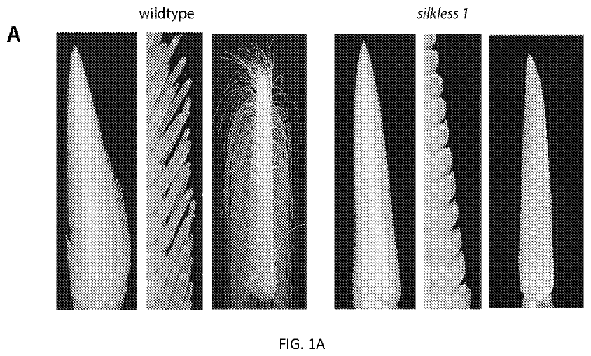

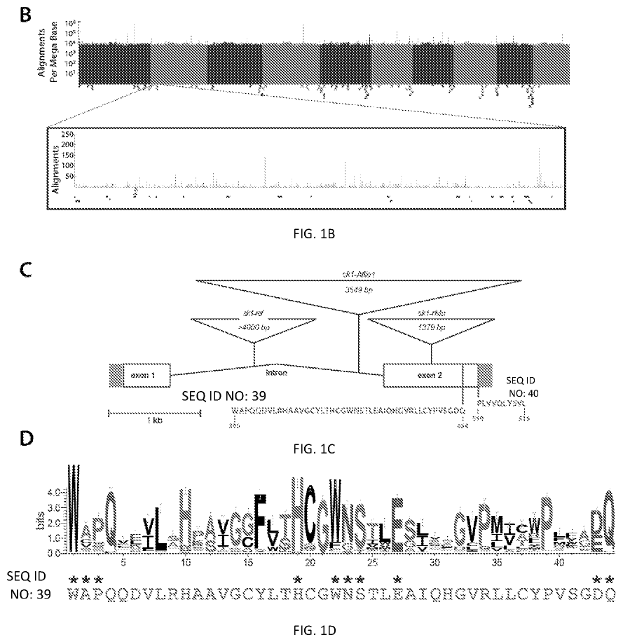

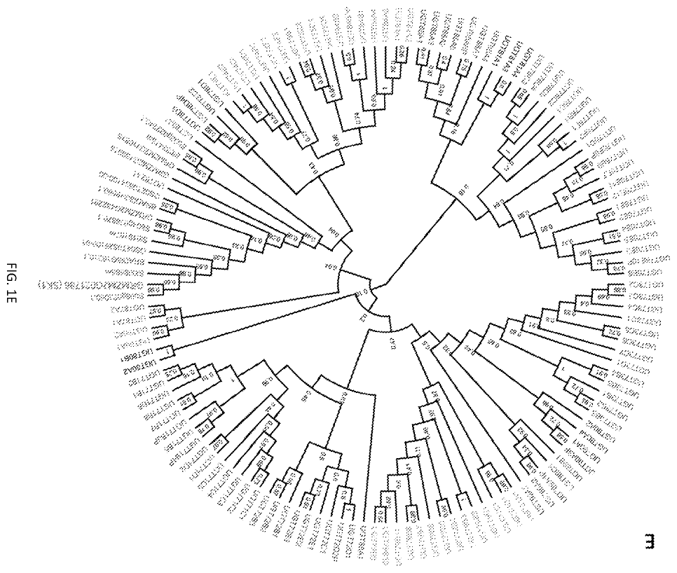

A third phylogenetic tree was developed to identify the relationship of SK1 and with 107 UGT proteins identified in Arabidopsis (FIG. 1E). The amino acid sequences of all proteins used in the second tree were aligned using ClustalW (Codon alignment, gap open penalty 3.0, gap extension penalty 1.8). Gaps in the sequence were visually identified and trimmed from the alignment. To build the final tree, the maximum likelihood algorithm in MEGA6 with 100 bootstraps was used with an LG amino acid substitution model with gamma distribution. ClustalW amino acid sequence alignment of SK1 (GRMZM2G021768) to the nearest Arabidopsis homolog UGT82A1 (AT3G22250) is shown in FIG. 5C.

### Fluorescent Protein Fusion Constructs

Citrine:SVL (pYU2969) was created by fusing the coding sequence (CDS) of the last 10 AA of the SK1 protein (“−SVL” domain”) to the 3′-end of the Citrine CDS. SK1ΔSVL:Citrine:SVL (pYU2996) was created by fusing the full length SK1 CDS, excluding the −SVL domain, to the 5′-end of pYU2969. SK1:Citrine (pYU3103) and Citrine:SK1 (pYU3119) contained 3′- or 5′-end fusions of Citrine to the full length SK1 CDS. For all four constructs, these coding sequences were placed under control of the single CaMV 35S promoter with a tobacco etch viral (TEV) 5′ leader and the 35S terminator. These expression cassettes were then cloned into the plant expression vector pPZP200 described in P. Hajdukiewicz, Z. Svab, P. Maliga, The small, versatile pPZP family of Agrobacterium binary vectors for plant transformation. Plant Mol. Biol. 25, 989-994 (1994). Plasmid construction details available upon request. The peroxisomal marker used in this paper (peroxisome-mCherry) was obtained from the Arabidopsis Biological Resource Center (ABRC) stock CD3-983 described in B. K. Nelson, X. Cai, A. Nebenfuhr, A multicolored set of in vivo organelle markers for co-localization studies in Arabidopsis and other plants. Plant J. 51, 1126-1136 (2007).

### Transient Expression by Agroinfiltration

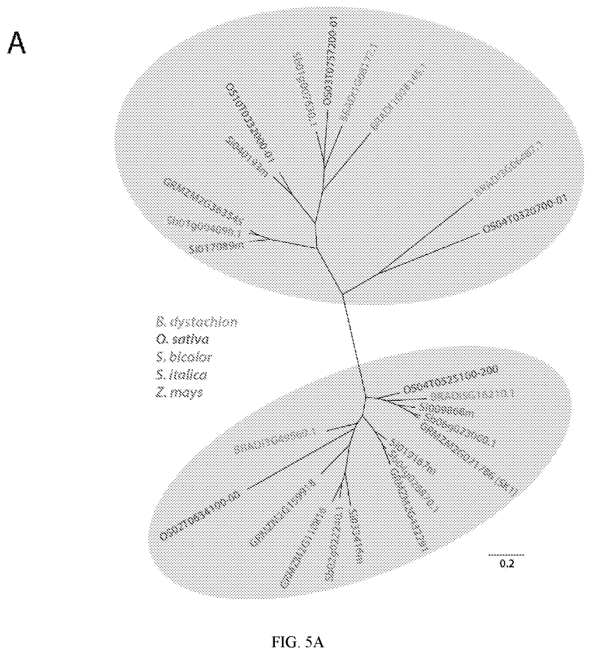

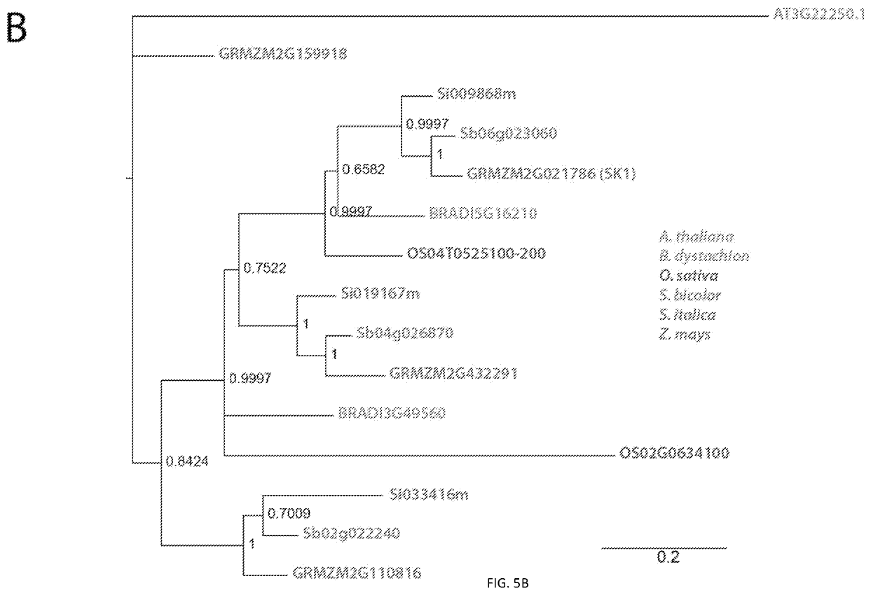

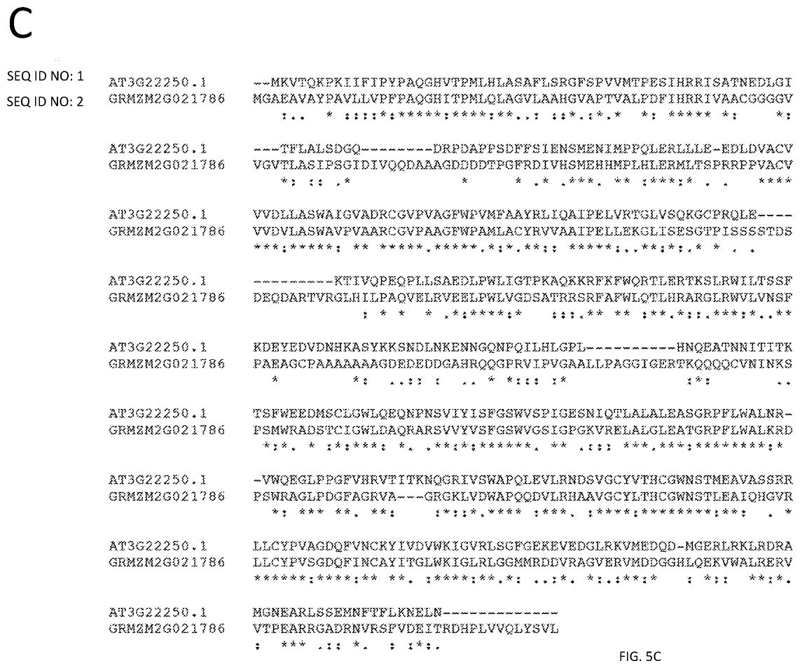

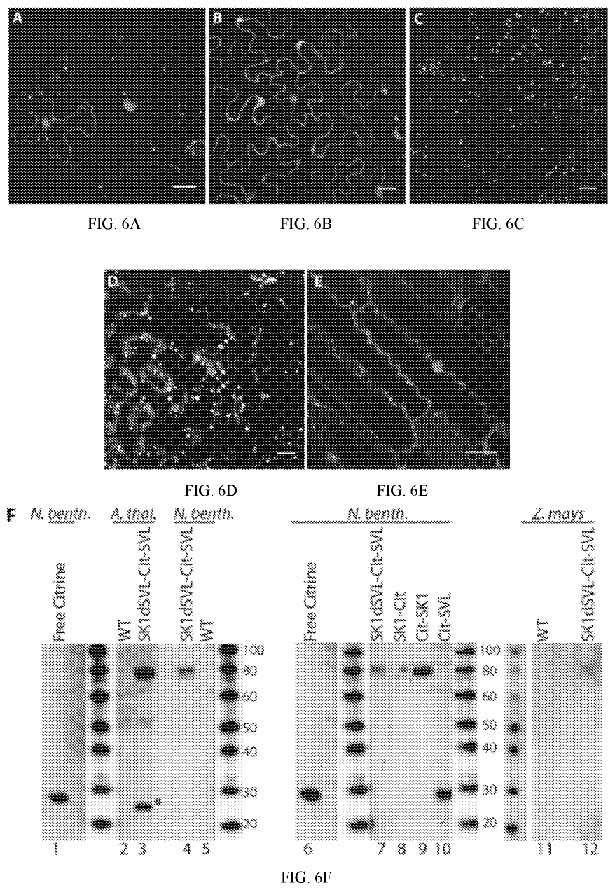

GV2260 Agrobacterium containing expression vectors were grown as previously described in A. Hayward, M. Padmanabhan, S. P. Dinesh-Kumar, Virus-induced gene silencing in Nicotiana benthamiana and other plant species. Methods Mol. Biol. 678, 55-63 (2011). Briefly, Agrobacterium was grown overnight, pelleted, and resuspended in infiltration medium containing 10 mM MgCl2, 10 mM 2-morpholinoethanesulfonic acid and 200 mM acetosyringone. Strains were induced at room temperature for 4 hours followed by vacuum infiltration into 4-5 week old N. benthamiana leaves at OD600 1.2-1.4. For co-infiltration, equal volumes of Agrobacterium were mixed at OD600=1.6-1.8. A further 1:10 or 1:100 dilution of Agrobacterium in infiltration medium prior to infiltration was sometimes used to produce optimum expression levels for confocal microscopy. All fusion proteins expressed transiently in N. benthamiana tissue were confirmed by western blotting (FIG. 6F).

### Fluorescence Microscopy

Live tissue microscopy was performed on a Zeiss LSM510 META confocal microscope (CARL ZEISS™) using a 40× C-Apochromat water immersion objective lens. For transient expression experiments, tissue samples were cut from N. benthamiana leaves at approximately 42 hours post infiltration. Transgenic Arabidopsis, N. benthamiana, or maize leaves were sampled from 3-6 week old plants. The 488 nm laser line of a 25 mW argon laser (COHERENT™) with BP 500-550 IR emission filter was used to image Citrine and the same laser line with META detector (651-683 nm) was used to image chloroplasts. The 561 nm laser line of a DPSS laser with BP 575-630 IR emission filter was used to image mCherry.

### Analysis of Sk1 Gene Expression in Maize Tissues

Expression of sk1-B73 was determined by an in silico analysis of twenty-four RNA-seq samples from eight distinct tissue types—stem shoot apical meristem, anthers, immature tassel, meiotic tassel, immature cob, pre-pollination cob, primary root, and eighth leaf (Table 2). RNA-seq data were acquired from NCBI Short Read Archive study SRP014652. This study was selected due to the availability of three replicates for each tissue. Reads were initially aligned to Zea mays AGP v. 3.22 reference genome then counted against Zea mays v. 3.22 transcriptome annotation using the TopHat pipeline default parameters with—b2—very-sensitive option. Read counts were obtained with HTSeq with default parameters. Read counts were normalized via EdgeR and normalized pseudocounts were used for analysis.

### Generation of Transgenic Plants

To generate stable transgenic SK1ΔSVL:Citrine:SVL Arabidopsis, pYU2996 was first transformed into Agrobacterium strain GV3101. Transgenic Arabidopsis lines were then generated using the floral dip method described in X. Zhang, R. Henriques, S. S. Lin, Q. W. Niu, N. H. Chua, Agrobacterium-mediated transformation of Arabidopsis thaliana using the floral dip method. Nat. Protoc. 1, 641-646 (2006). Arabidopsis transformants were selected by 0.02% BASTA spray (Finale©). Stable transgenic SK1ΔSVL:Citrine:SVL N. benthamiana plants were generated as described in T. Clemente, Nicotiana (Nicotiana tobaccum, Nicotiana benthamiana). Methods Mol. Biol. 343, 143-154 (2006) with modifications to the media as described below. Agrobacterium cultures were grown in LB medium with appropriate antibiotics. Cocultivation medium contained 1/10 MS basal media and vitamins, 30 mM MES, 3% sucrose, 1 μg/mL BAP, 100 ng/mL NAA, and 200 μM acetosyringone. Selection medium contained 1× MS basal media and vitamins, 3% sucrose, 1 μg/mL BAP, 100 ng/mL NAA, 500 μg/mL Timentin, and 3 μg/mL glufosinate. Rooting medium contained ½ MS basal media and vitamins, 1% sucrose, 100 ng/mL NAA, 500 μg/mL Timentin, and 3 μg/mL glufosinate. To generate SK1ΔSVL:Citrine:SVL maize transgenics, pYU2996 was moved into Agrobacterium strain EHA101 via electroporation. Sixty-three independent transgenic maize events were produced following the procedure of J. M. Vega, W. Yu, A. R. Kennon, X. Chen, Z. J. Zhang, Improvement of Agrobacterium-mediated transformation in Hi-II maize (Zea mays) using standard binary vectors. Plant Cell Rep. 27, 297-305 (2008) with modifications to the media described below. Plant tissue culture grade agar (8 g/L) was used in place of Gelrite until plant regeneration. To eliminate Agrobacteria post co-cultivation, 150 mg/L carbenicillin was used in conjunction with 100 mg/L vancomycin instead of cefotaxime. During plant regeneration, 100 mg/L myo-inositol was added to the medium and 3 mg/L bialaphos was maintained until transplantation. SK1ΔSVL:Citrine:SVL expression in stable transgenic Arabidopsis and N. benthamiana, and maize was confirmed by western blotting (FIG. 6F).

### Screening of Transgenic SK1ΔSVL:Citrine:SVL Maize

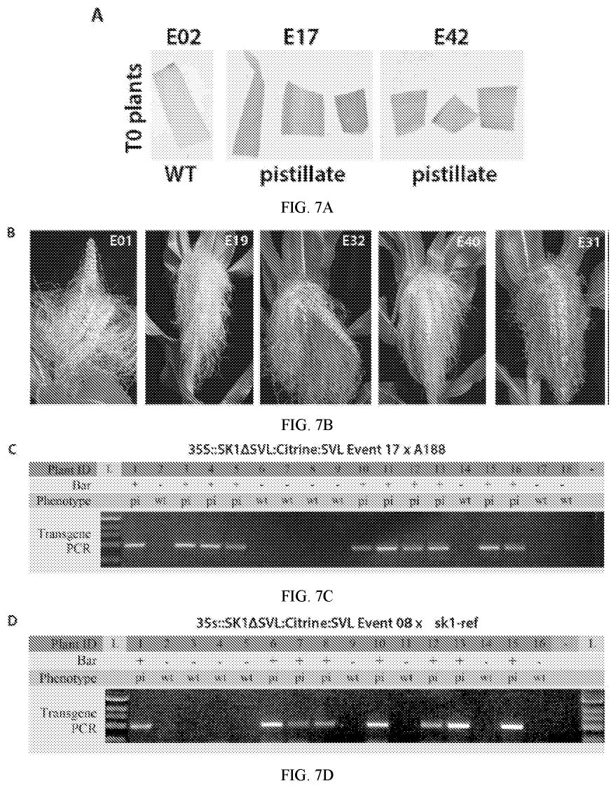

Transgenic maize plants were screened for presence of the transgene cassettes via swabbing of mature leaves with 3% Finale® herbicide according to W. J. Gordon-Kamm et al., Transformation of Maize Cells and Regeneration of Fertile Transgenic Plants. The Plant cell 2, 603-618 (1990). Resistance or sensitivity to the herbicide was scored after 4 days. Leaves of T0 plants were screened for SK1ΔSVL:Citrine:SVL transgene expression using the 532 nm laser line of a Typhoon 9400 fluorescence imager with 526 SP filter. A subset of T1 plants were further screened for the presence of the SK1ΔSVL:Citrine:SVL transgene by PCR assay with primers targeting either A) the 3′ end of the SK1ΔSVL:Citrine:SVL coding sequence including the 35S terminator or B) the bar selectable marker (FIG. 7H-7I). PCRs were performed using Phusion DNA polymerase (NEW ENGLAND BIOLABS®).

### Monitoring Protein Levels

Plant tissue expressing the proteins of interest was collected and ground in liquid nitrogen. Protein was extracted with buffer containing 50 mM NaCl, 20 mM Tris/HCL pH 7.5, 1 mM EDTA pH 8.0, 0.75% Triton X-100, 10% glycerol, 2 mM DTT, 4 mM NaF, 2 mM PMSF, and Complete Protease Inhibitors (ROCHE®). To facilitate detection of SK1-Citrine in SK1:Citrine:SVL maize leaf tissue, crude immunoprecipitation was performed to concentrate the protein using GFP-nAb magnetic beads (ALLELE®). The appropriate volume of 2×SDS loading buffer was added to each sample, and samples were heated at 90° C. for 10 minutes prior to loading. Protein was run on polyacrylamide gels and transferred to PVDF membrane (MILLIPORE®) for Western blot analysis and Citrine fusions were detected using mouse anti-GFP (COVANCE®) and rabbit anti-mouse-HRP (SIGMA®).

### Quantification of Jasmonates in Terminal Inflorescences

The developing terminal inflorescence of SK1ΔSVL:Citrine:SVL T1 maize plants of +/+ and SK1-CIT/+ genotypes were dissected between 2.5 and 13 cm in length and rapidly frozen in liquid nitrogen. Tissue samples were stored at −80° C. prior to metabolite extraction. Jasmonate quantification was performed as described in W. J. Gordon-Kamm et al., Transformation of Maize Cells and Regeneration of Fertile Transgenic Plants. The Plant cell 2, 603-618 (1990) with minor modifications. Briefly, plant tissues were ground under liquid nitrogen and 200 mg of fresh frozen powder was weighed in microcentrifuge tubes. To the tubes were added 1.5 mL of acidified isopropanol, 10 μL of internal standard (d5-JA) and 5-10 glass beads. Extraction was performed in a paint shaker for 3 min, followed by centrifugation and evaporation to dryness. The extract was purified by solid phase extraction (SPE), dried again and reconstituted in 300 μL, of methanol:H2O (85:15, v/v) prior to analysis. Jasmonate profiling was achieved by ultra-high pressure liquid chromatography coupled to high resolution mass spectrometry. Concentrations of jasmonates were calculated by normalizing the obtained peaks to that of the internal standard.

### Sequences

TABLE 2

| Primer | Primer sequence | |
|---|---|---|
| ID | (listed 5′ to 3′) | Purpose |
| 1811 | SEQ ID NO: 3 | Amplification of SSR marker AY107034 for |
| | AAAGTGTCCTGGCTTGCAG | mapping of the sk1 locus |
| | ATACC | |
| 1825 | SEQ ID NO: 4 | Amplification of SSR marker AY107034 for |
| | AAGCATTCTAGGGCACACA | mapping of the sk1 locus |
| | TTGAT | |
| 1655 | SEQ ID NO: 5 | Amplification of SSR marker b1 (umc1776) for |
| | AAGGCTCGTGGCATACCTG | mapping of the sk1 locus* |
| | TAGT | |
| 1656 | SEQ ID NO: 6 | Amplification of SSR marker b1 (umc1776) for |
| | GCTGTACGTACGGGTGCAA | mapping of the sk1 locus* |
| | TG | |
| 782 | SEQ ID NO: 7 | Amplification of indel marker bnl8.04 for |
| | GTCATCACTCATCAATCCC | mapping of the sk1 locus |
| | AGC | |
| 783 | SEQ ID NO: 8 | Amplification of indel marker bnl8.04 for |
| | TCAACCCCCACCTCTCTATT | mapping of the sk1 locus |
| | TATA | |
| 773 | SEQ ID NO: 9 | Amplification of CAPS (HaeIII) marker |
| | CCTACCCGCTACAACTGGA | bnl12.09 for mapping of the sk1 locus |
| | CATAA | |
| 781 | SEQ ID NO: 10 | Amplification of CAPS (HaeIII) marker |
| | CAGTACTCGTTTGTGCAGTT | bnl12.09 for mapping of the sk1 locus |
| | TGCT | |
| 1573 | SEQ ID NO: 11 | Amplification of SSR marker bnlg1064 for |
| | CTGGTCCGAGATGATGGC | mapping of the sk1 locus* |
| 1574 | SEQ ID NO: 12 | Amplification of SSR marker bnlg1064 for |
| | TCCATTTCTGCATCTGCAAC | mapping of the sk1 locus* |
| 1571 | SEQ ID NO: 13 | Amplification of SSR marker phi109642 for |
| | CTCTCTTTCCTTCCGACTTT | mapping of the sk1 locus* |
| | CC | |
| 1572 | SEQ ID NO: 14 | Amplification of SSR marker phi109642 for |
| | GAGCGAGCGAGAGAGATC | mapping of the sk1 locus* |
| | G | |
| 1621 | SEQ ID NO: 15 | Amplification of SSR marker umc1555 for |
| | ATAAAACGAACGACTCTCT | mapping of the sk1 locus* |
| | CACCG | |
| 1622 | SEQ ID NO: 16 | Amplification of SSR marker umc1555 for |
| | ATATGTCTGACGAGCTTCG | mapping of the sk1 locus* |
| | ACACC | |
| 1727 | SEQ ID NO: 17 | Amplification of indel marker umc1769 for |
| | GACGCGACTTATTCAGCAC | mapping of the sk1 locus |
| | CAC | |
| 1733 | SEQ ID NO: 18 | Amplification of indel marker umc1769 for |
| | ATTGTTTCAGCGCTGCCGG | mapping of the sk1 locus |
| | TTA | |
| 661 | SEQ ID NO: 19 | Amplification of indel marker umc34 for |
| | CAACTTCGAGGCAGTTCGT | mapping of the sk1 locus |
| | TTAT | |
| 662 | SEQ ID NO: 20 | Amplification of indel marker umc34 for |
| | AGCTCTTGTTGCAGGAAGT | mapping of the sk1 locus |
| | AGGAC | |
| 2159 | SEQ ID NO: 21 | Amplification of CAPS (Mwo1) marker |
| | GCGTTGTTTGGTAGATCGTT | FG12180 |
| | AGCC | |
| 2160 | SEQ ID NO: 22 | Amplification of CAPS (Mwo1) marker |
| | CATATGCATCAGGTCAAGC | FG12180 |
| | AAGGA | |
| 2180 | SEQ ID NO: 23 | Amplification of CAPS (SacII) marker FG06631 |
| | ACTGCATCTCACTTGTCACC | |
| | GTCT | |
| 2187 | SEQ ID NO: 24 | Amplification of CAPS (SacII) marker FG06631 |
| | TGCAGCTTAAATTTCATGG | |
| | ACGTG | |
| 2205 | SEQ ID NO: 25 | Amplification of CAPS (BsiHKAI) marker |
| | GCCGAGGATTTCCTGCTGA | FG06659 |
| | AG | |
| 2206 | SEQ ID NO: 26 | Amplification of CAPS (BsiHKAI) marker |
| | GCTCATGTTGCTTCACAAC | FG06659 |
| | CTCTC | |
| TA_BC1F | SEQ ID NO: 27 | Forward adapter used to create the ski Taq^{α}I |
| | ACACTCTTTCCCTACACGA | library for plant P19-33 |
| | CGCTCTTCCGATCTAGCTT | |
| TA_BC1R | SEQ ID NO: 28 | Reverse adapter used to create the sk1 Taq^{α}I |
| | [Phos]AGCTAGATCGGAAGA | library for plant P19-33 |
| | GCGTCGTGTAGGGAAAGAG | |
| | TG | |
| TA_BC2F | SEQ ID NO: 29 | Forward adapter used to create the sk1 Taq^{α}I |
| | ACACTCTTTCCCTACACGA | library for plant P22-24 |
| | CGCTCTTCCGATCTGCTAT | |
| TA_BC2R | SEQ ID NO: 30 | Reverse adapter used to create the sk1 Taq^{α}I |
| | [Phos]TAGCAGATCGGAAGA | library for plant P22-24 |
| | GCGTCGTGTAGGGAAAGAG | |
| | TG | |
| TA_BC3F | SEQ ID NO: 31 | Forward adapter used to create the sk1 Taq^{α}I |
| | ACACTCTTTCCCTACACGA | library for plant P4-48 |
| | CGCTCTTCCGATCTCTAGT | |
| TA_BC3R | SEQ ID NO: 32 | Reverse adapter used to create the sk1 Taq^{α}I |
| | [Phos]CTAGAGATCGGAAGA | library for plant P4-48 |
| | GCGTCGTGTAGGGAAAGAG | |
| | TG | |
| TA_BC4F | SEQ ID NO: 33 | Forward adapter used to create the sk1 Taq^{α}I |
| | ACACTCTTTCCCTACACGA | library for plant P13-27 |
| | CGCTCTTCCGATCTGATGT | |
| TA_BC4R | SEQ ID NO: 34 | Reverse adapter used to create the sk1 Taq^{α}I |
| | [Phos]CATCAGATCGGAAGA | library for plant P13-27 |
| | GCGTCGTGTAGGGAAAGAG | |
| | TG | |
| CommonF | SEQ ID NO: 35 | Forward adapter used to create the sk1 Taq^{α}I |
| | CTCGGCATTCCTGCTGAAC | library for all four sk1 plants |
| | CGCTCTTCCGATCT | |
| CommonR | SEQ ID NO: 36 | Reverse adapter used to create the sk1 Taq^{α}I |
| | [Phos]GATCGGAAGAGCGGT | library for all four sk1 plants |
| | TCAGCAGGAATGCCGAG | |
| Buc | SEQ ID NO: 37 | Primer used for Illumina ® library |
| PCR1 | AATGATACGGCGACCACCG | amplification |
| | AGATCTACACTCTTTCCCTA | |
| | CACGACGCTCTTCCGATCT | |
| Buc | SEQ ID NO: 38 | Primer used for Illumina ® library |
| PCR2 | CAAGCAGAAGACGGCATAC | amplification |
| | GAGATCGGTCTCGGCATTC | |
| | CTGCTGAACCGCTCTTCCG | |
| | ATCT | |

The results of the experiments are now described.

### Example 1

The only functional pistils in most lines of maize are found in the primary ear florets. The presence of these functional pistils requires the action of the silkless 1 (sk1) gene. In sk1 mutant plants all pistils are eliminated (FIG. 1A), a phenotype dependent on the action of the ts1 and ts2 genes. The epistasis between the is genes and sk1 suggests that sk1 functions to protect the pistils from the JA-mediated elimination signal encoded by ts1 and ts2 genes.

To investigate this model for sk1 activity, the maize sk1 gene was identified using a positional interval mapping and next generation sequencing (NGS) approach. A genetic (0.2 cM) and physical (700 kb) interval containing the sk1 gene was defined using recombination mapping in an F2 population segregating for the sk1 reference allele (sk1-ref) (FIG. 4A). A candidate sk1 gene was identified within this interval by the characterization of a second sk1 allele (sk1-rMu1) derived from active Mutator (Mu) maize lines. Mu-Taq, a genome sequencing approach that enriches for Mu-chromosomal junction fragments was utilized. Of the 179 total independent Mu junction fragments identified, two mapped within the coding sequence of GRMZM2G021786, a predicted gene located within the sk1 genetic interval, making it a candidate for the sk1 gene (FIG. 1B). The sk1-mu1 allele contained a 1379 bp insertion 98% identical to the canonical Mu1 element in the second predicted exon of GRMZM2G021786 (FIG. 1C) (Table 1).

To verify GRMZM2G021786 is sk1, independent sk1 mutant alleles were examined. In the sk1-ref allele a Helitron-like transposable element was identified in the intron of GRMZM2G021786 (FIG. 1C). The insertion in sk1 lacked terminal inverted repeats, did not cause a target site duplication and inserted between the dinucleotide motif AT, characteristics of other Helitron-induced mutations in maize. A third independent allele, sk1-Allie1 contained a novel 3,549 bp insertion in the intron of GRMZM2G021786 (FIG. 1C). Together, these results provide independent confirmation of the identity of GRMZM2G021786 as the sk1 gene.

The sk1 gene encodes a 512 AA protein with high similarity to family 1 UDP-glycosyltransferases (UGT) (FIGS. 1CD, 4). Alignment of the SK1 protein to 107 identified Arabidopsis UGTs confirmed the presence of a plant secondary product glycosyltransferase (PSPG) box at AA384-AA434, a conserved motif that is a defining feature of plant UGTs (FIGS. 1C,D). The SK1 PSPG box contains seven conserved amino acids at positions shown to form hydrogen bonds to invariant parts of the UDP-sugar donor in structural studies of other plant UGTs (FIG. 1D). Three other positions, W22, D43, and Q44, are also conserved and have been shown to interact with the variable UDP-sugar moieties of both UDP-galactose and UDP-glucose donor molecules. A putative peroxisomal targeting sequence (PTS) was identified at the C-terminus of SK1 (“−SVL”) that shows some similarity to the canonical −SKL PTS1 motif (FIG. 1C). When compared to all known and predicted Arabidopsis UGTs, SK1 exhibited the greatest similarity to UGT82A1 encoded by At3g22250 (E value=1e-131 with 43% identity) the sole member of the biochemically uncharacterized Arabidopsis UGT Group N (FIG. 1E).

### Example 2

All plant UGTs catalyze the transfer of donor uridine diphosphate-activated sugars (e.g. UDP-glucose) to diverse small molecule acceptor substrates. Phytohormones and secondary metabolites have been identified as targets of plant UGT activity. In vivo studies have shown that auxin, brassinosteroids, salicylic acid, flavonoids, and glucosinolates can all serve as endogenous UGT-acceptors. The inhibition of phytohormone signaling by glycosylation has been commonly described, with the glycosylated substrates undergoing sequestration or catabolism to prevent further activity. Since sk1 inhibits JA-dependent pistil abortion, its glycosyltransferase activity might inactivate JA or one of its precursors known to be synthesized in peroxisomes to disrupt JA signaling and tasselseed-mediated pistil elimination.

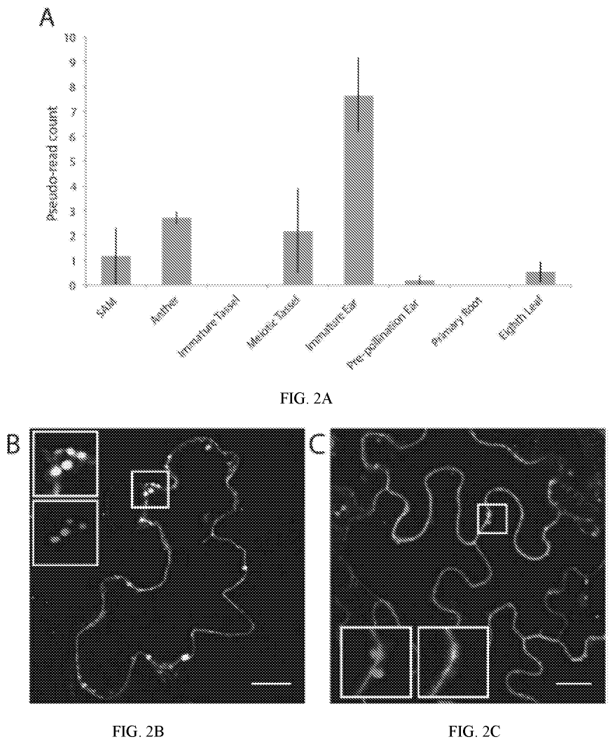

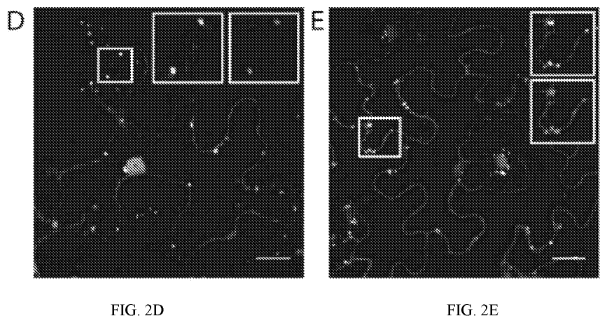

To further investigate the function of sk1, expression and localization, studies were conducted. A meta-analysis of RNA-seq data for GRMZM2G021786 revealed extremely low expression across all tissues probed, with no individual sample exceeding a read pseudo-count often (FIG. 2A). Consistent with its role in protecting ear pistils, the highest sk1 expression was observed in the immature ear (mean read count of 7.66±1.50 (SE)), a time at which pistil protection takes place. Perhaps due to its extremely low expression, the SK1 RNA was undetectable by in situ hybridization. Next the localization of the SK1 protein and the role of the putative PTS located at the C-terminus of SK1 (“−SVL”) was examined. A fusion of the last ten amino acids of the SK1 protein, which included the −SVL tripeptide, to the C-terminus of the Citrine fluorescent protein (Citrine: SVL) was sufficient to localize Citrine to plant peroxisomes during transient overexpression in Nicotiana benthamiana tissue (FIG. 2B; FIG. 6A). A fusion of Citrine to the C-terminus of the full-length SK1 protein (SK1:Citrine), however, did not show peroxisomal localization, presumably because the −SVL localization signal was blocked (FIG. 2C, FIG. 6B). When the putative PTS domain was relocated to the C-terminus of the SK1-Citrine protein fusion constructs (SK1:Citrine:SVL or Citrine:SK1) localization to plant peroxisomes was restored (FIG. 2D-E). The localization pattern of SK1:Citrine:SVL was confirmed in the leaf tissue of stable transgenic Arabidopsis (FIG. 6C), N. benthamiana (FIG. 6D), and maize (FIG. 6E). Together these results confirm that the SK1 protein localizes to plant peroxisomes by a requisite C-terminal PTS1-like motif.

### Example 3

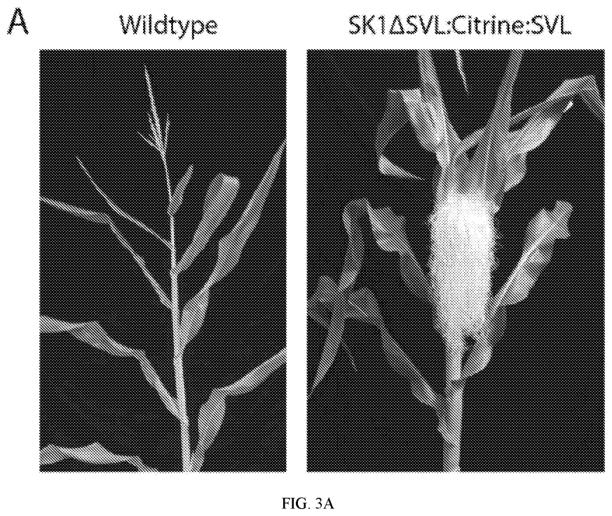

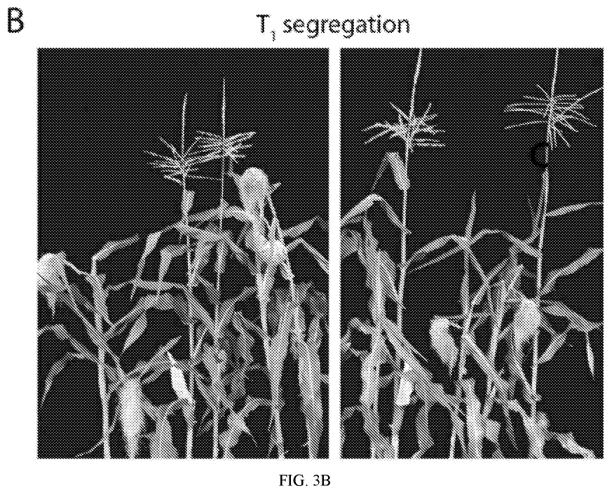

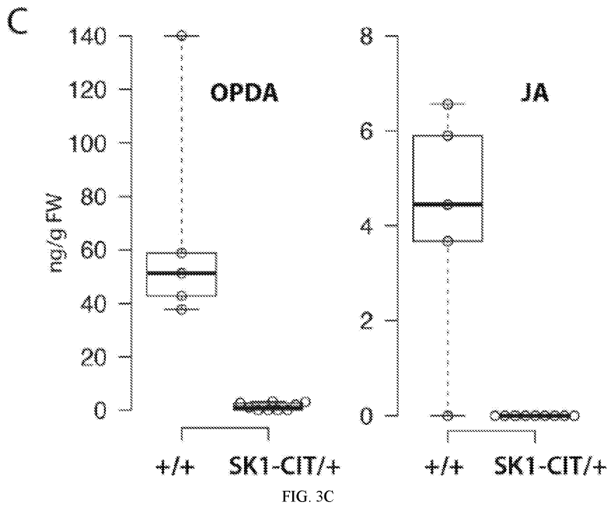

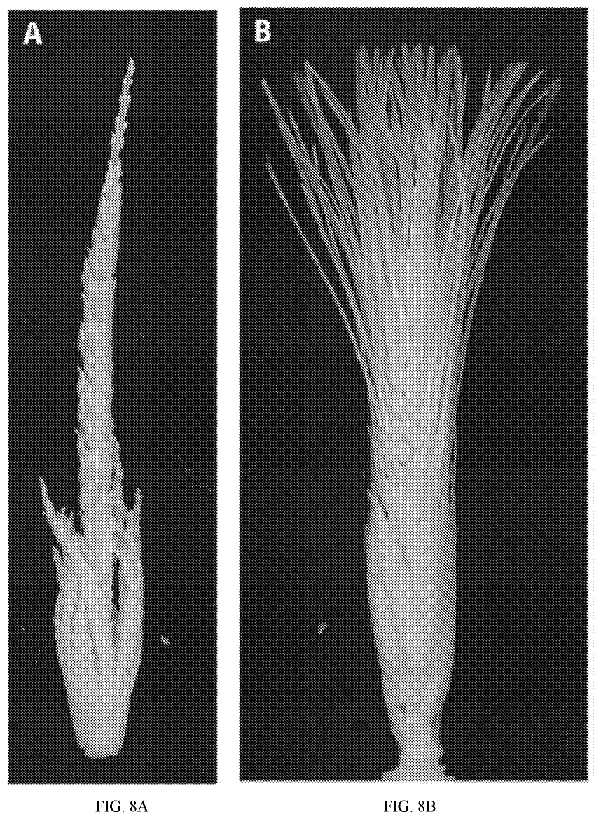

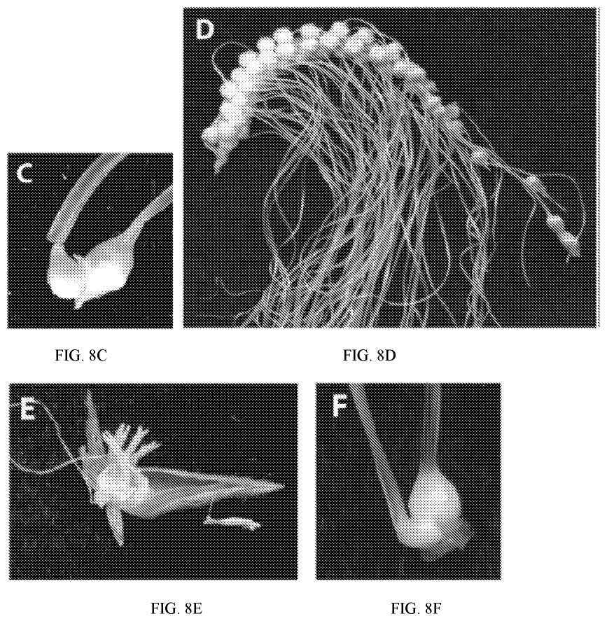

Genetic analysis has shown that sk1 is required to protect functional pistils in ear spikelets from tasselseed-mediated elimination. The elimination of all pistils in the tassel and of the secondary ear pistils requires a functional ts1 and ts2 gene and in ts1 and ts2 mutant plants all pistils in the plant fail to abort. In order to test whether ectopic sk1 expression could protect pistils destined to be eliminated by tasselseed action, maize plants were transformed and regenerated with an sk1 transgene (SK1:Citrine:SVL) driven by a constitutive CaMV 35S promoter (FIG. 7). In transgenic 35S:SK1:Citrine:SVL maize the SK1-Citrine fusion protein localized to punctate bodies (FIG. 6E), mirroring the localization described during heterologous expression. A total of 72 primary transgenic plants (T0) representing 18 independent transformation events were characterized after scoring positively for transgene expression (FIG. 7A). All 72 T0 plants displayed complete feminization of the tassel inflorescence (pistillate tassels) and double ear pistils indicating that all pistils were protected from elimination (FIG. 3A, FIG. 7B, FIG. 8F). One T0 plant from a non-productive transgenic event (negative for Citrine fluorescence) produced a wild type staminate tassel. T0 plants representing 13 independent events were crossed by wild type males. As expected, most T1 families segregated for the presence of the transgene as determined by PCR and sensitivity to the herbicide phosphinothricin encoded by the selectable marker bar used in the transformation vector (FIG. 7C-D). 226 of 228 bar positive plants displayed pistillate tassels while 182 of 182 bar negative plants were wild type with staminate tassels (FIG. 3B). The T0 and T1 pistillate tassel phenotype was highly penetrant and expressive in transgenic 35S:SK1:Citrine:SVL maize, with all tassel spikelets displaying complete feminization (FIG. 8A-C). Partial expressivity was only rarely observed among hundreds of plants in a few most apical spikelets as the presence of rudimentary anthers (FIG. 8D-E). Together these results indicate that sk1 expression is sufficient to block the tasselseed-mediated elimination of pistils in both ear and tassel spikelets resulting in a completely feminized plant.

### Example 4

The tasselseed genes eliminate pistils by stimulating the production of jasmonates. Therefore, it was investigated whether the protection mediated by sk1 was accompanied by altered JA levels. Jasmonate levels were examined in both wild-type and pistillate tassels of T1 plants segregating 1:1 for the SK1:Citrine:SVL transgene. As expected, JA and its precursor molecule 12-oxo-phytodienoic acid (OPDA) were readily detected in developing staminate tassels that did not express the sk1 transgene (+/+; FIG. 3C). However, OPDA was strongly reduced (˜50-fold) and JA was undetectable in sibling transgenics with pistillate tassels (SK1-CIT/+). These results indicate that sk1 expression strongly attenuates JA levels and its immediate precursor OPDA implying a mechanism of sk1 protection by preventing JA-mediated pistil elimination.

JA signaling is attenuated by catabolism of biologically active JA-L-isoleucine via cytochrome P450 hydroxylases or IAA amidohydrolases localized in the endoplasmic reticulum. Such attenuation may prevent the persistence of costly stress-activated JA responses. The homology of SK1 to UGTs, another type of small-molecule-modifying enzymes, raises the possibility of another mechanism for JA signaling control in the developmental process of floral sexuality, in this case through the modification of JA synthesis intermediaries localized in the peroxisomes.

Untargeted metabolite profiling was performed using high resolution mass spectrometry to attempt to identify modified JA intermediates specific to SK1-Citrine activity in SK1:Citrine:SVL tassels, but were unable to identify a putative SK1 target in these experiments. The low native expression level of sk1 at the developing ear (FIG. 2A) suggests that only a small amount of SK1 may be required for inhibition of JA signaling, and an SK1-dependent intermediate may exist below the detection limits. However, the possibility that SK1-Citrine acts upstream of JA biosynthesis, and that the changes in OPDA and JA levels in SK1:Citrine:SVL tassels are an indirect consequence of SK1-mediated sex determination cannot be excluded.

Maize is one of several grasses with a sex determination system that results in imperfect florets. Yet, many related grasses such as sorghum bear perfect rather than imperfect florets. Four of these related grasses with complete genome sequences available, Brachypodium distachyon, Oryza sativa, Setaria italica, and Sorghum bicolor were examined for potential sk1 orthologs. Single copy orthologs of sk1 were identified in each of these four grasses even though they possess perfect florets (FIG. 5). The orthologs of sk1 were analyzed for the presence of a PTS1 domain similar to the −SVL domain required for maize sk1 to localize to peroxisomes. The C-terminal −SVL tripeptide was also found in the S. bicolor sk1, while another previously reported PTS1 sequence, −STL, was identified in B. distachyon sk1. No PTS1 or PTS1-like sequence could be identified in the S. italica or O. sativa sk1 orthologs. Only one other UGT, the sterol glucosyltransferase UGT51 (ATG26), has previously been shown to localize to peroxisomes where it has been shown to promote peroxisomal degradation by autophagy. UGT51 does not have a PTS motif, instead associating with the peroxisome membrane via protein-protein interactions with the micropexophagic apparatus. No homologs of UGT51 have been identified outside of yeast and UGT51 does not show significant homology to SK1.

This study shows that the simple segregation of a gain-of-function sk1 transgene can be used to effectively control sexuality in maize. When this transgene is expressed in the sk1 mutant background, production of staminate and pistillate maize plants can be stably maintained even in open pollinated field conditions. Moreover, the physical linkage of an herbicide resistance trait to the sk1 transgene can be used to completely feminize a maize population by herbicide application in the field.

Although the present invention has been described in detail with reference to examples above, it is understood that various modifications can be made without departing from the spirit of the invention. Accordingly, the invention is limited only by the following claims. All cited patents and publications referred to in this application are herein incorporated by reference in their entirety.

1. An isolated polynucleotide encoding a polypeptide of SEQ ID NO: 2 or an amino acid sequence variant thereof operably linked to a heterologous promoter.

2. The isolated polynucleotide of claim 1, wherein the heterologous promoter is a CaMV 35S promoter.

3. The isolated polynucleotide of claim 1 further comprising a marker gene.

4. The isolated polynucleotide of claim 3, wherein the marker gene is an herbicide resistance gene.

5. The isolated polynucleotide of claim 4, wherein the herbicide resistance gene is bar.

6. The isolated polynucleotide of claim 4, wherein the herbicide resistance gene encodes 5-enolpyruvyl-shikimate synthase (ESPS).

7. The isolated polynucleotide of claim 3, wherein the marker gene affects the visual appearance of the seed or seedling.

8. The isolated polynucleotide of claim 7, wherein the marker gene controls the appearance or distribution of anthrocyanin pigments in the seed or seedling.

9. A plant cell transformed with the isolated polynucleotide of claim 1.

10. A genetically modified plant comprising a transgene containing an sk1-encoded glycosyltransferase operably linked to a promoter for heterologous expression in the cells of the plant.

11. The genetically modified plant of claim 10, wherein the plant is maize, sorghum or rice.

12. The genetically modified plant of claim 10, wherein the genetically modified plant is a unisexual plant.

13. A genetically modified plant comprising a transgene encoding a uridine diphosphate (UDP) glycosyltransferase.

14. The genetically modified plant of claim 13, wherein the plant is maize, sorghum or rice.

15. The genetically modified plant of claim 14, wherein the genetically modified plant comprises inflorescences of the pistillate phenotype associated with sk1.

16. The genetically modified plant of claim 15, wherein the inflorescences are solely of the pistillate phenotype associated with sk1.

17. A genetically modified plant comprising a mutation or transgene targeting an endogenous UDP glycosyltransferase and disrupting its activity.

18. The plant of claim 17, wherein the UDP glycosyltransferase is sk1.

19. The genetically modified plant of claim 17, wherein the plant is maize, sorghum or rice.

20. The genetically modified plant of claim 17 comprising inflorescences of the staminate phenotype associated with the disruption of sk1.

21. The genetically modified plant of claim 17, wherein the genetically modified plant is a unisexual plant.

22. The genetically modified plant of claim 17, wherein the mutation is engineered using a CRISPR/Cas9 system.

23. A method of generating a genetically modified plant comprising transforming a cell with a construct comprising a transgene encoding a UDP glycosyltransferase, thereby promoting the expression of the UDP glycosyltransferase in one or more cells of the plant.

24. The method of claim 23, wherein the transgene is sk1.

25. The method of claim 23, wherein the transgene comprises a polynucleotide encoding a polypeptide of SEQ ID NO: 2 or an amino acid sequence variant thereof.

26. The method of any one of claim 23, wherein the transgene is operably linked to a heterologous promoter.

27. The method of claim 26, wherein the heterologous promoter is a CaMV 35S promoter.

28. The method of claim 23, wherein the UDP glycosyltransferase localizes to a peroxisome.

29. The method of any one of claim 23, wherein the construct further comprises a marker gene.

30. The method of claim 29, wherein the marker gene is an herbicide resistance gene.

31. The method of claim 30, wherein the herbicide resistance gene is bar.

32. The method of claim 30, wherein the herbicide resistance gene encodes 5-enolpyruvyl-shikimate synthase (ESPS).

33. The method of claim 29, wherein the marker gene affects the visual appearance of a seed or seedling.

34. The method of claim 33, wherein the marker gene controls the appearance or distribution of one or more anthrocyanin pigments in the seed or seedling.

35. The method according to claim 29, further comprising using the marker gene to select at least one genetically modified plant.

36. The method according to claim 35, further comprising using the genetically modified plant to generate a hybrid seed.

37. The method according to claim 29, wherein the plant is maize, rice or sorghum.

38. A method of generating a transgenic plant comprising the step of engineering a mutation or transgene targeting an endogenous UDP glycosyltransferase and disrupting its activity.

39. The method of claim 38, wherein the UDP glycosyltransferase is sk1.

40. The method of claim 38, wherein the plant is maize, sorghum or rice.

41. The method of claim 38, wherein the plant comprises at least one inflorescence of the staminate phenotype associated with the disruption of sk1.

42. The method of claim 40, wherein the wherein the transgenic plant is a unisexual plant.

43. The method of claim 38, wherein the mutation is engineered using a CRISPR/Cas9 system.

44. A method of generating a transgenic plant comprising engineering a mutation in a 5′ or 3′ regulatory element of an endogenous UDP glycosyltransferase to alter an expression level of the UDP glycosyltransferase.

45. The method of claim 44, wherein the transgenic plant is maize, rice or sorghum.

46. The method of claim 44, wherein the transgenic plant is a unisexual plant.

47. The method of any one of claim 44, wherein the mutation is engineered using a crispr/Cas9 system, zinc-finger nucleases or transcription activator-like effects.
